SEQUENCE LISTING (47 sequences)

SEQ ID NO: 1 - protein, 5 aa, Artificial Sequence
  Formula I
RXXXX

SEQ ID NO: 2 - protein, 9 aa, Artificial Sequence
  Formula II
RXKXRXKKK

SEQ ID NO: 3 - protein, 16 aa, Artificial Sequence
  n is 2, peptide is a branched dimer
RXKXRXKKKKXRXKXR

SEQ ID NO: 4 - protein, 16 aa, artificial sequence
  n is 3, a branched peptide, a trimer
RXKXRXKKKKXRXKXR

SEQ ID NO: 5 - protein, 9 aa, Artificial Sequence
  n is 4, branched peptide is a tetramer
RXKXRXKKK

SEQ ID NO: 6 - protein, 9 aa, artificial sequence
  artificial peptide
RXRKXRRKK

SEQ ID NO: 7 - protein, 9 aa, Artificial Sequence
  artificial peptide
RARKXRRKK

SEQ ID NO: 8 - protein, 10 aa, Artificial Sequence
  artificial peptide
RARKAARRKK

SEQ ID NO: 9 - protein, 8 aa, Artificial Sequence
  artificial peptide
RARKRRKK

SEQ ID NO: 10 - protein, 9 aa, Artificial Sequence
  artificial peptide
RGRKXRRKK

SEQ ID NO: 11 - protein, 10 aa, Artificial Sequence
  artificial peptide
RGRKGGRRKK

SEQ ID NO: 12 - protein, 10 aa, Artificial Sequence
  artificial peptide
RGRKGGRRKK

SEQ ID NO: 13 - protein, 9 aa, Artificial Sequence
  artificial peptide
RGRKGRRKK

SEQ ID NO: 14 - protein, 8 aa, Artificial Sequence
  artificial peptide
RGRKRRKK

SEQ ID NO: 15 - protein, 10 aa, Artificial Sequence
  artificial peptide
RGRKAARRKK

SEQ ID NO: 16 - protein, 9 aa, Artificial Sequence
  artificial peptide
RGRKARRKK

SEQ ID NO: 17 - protein, 18 aa, Artificial Sequence
  artificial peptide
RGRKGGRRKKRRGGKRGR

SEQ ID NO: 18 - protein, 16 aa, Artificial Sequence
  artificial peptide
RGRKGRRKKRRGKRGR

SEQ ID NO: 19 - protein, 14 aa, Artificial Sequence
  artificial peptide
RGRKRRKKRRKRGR

SEQ ID NO: 20 - protein, 7 aa, Artificial Sequence
  artificial peptide
RRKRRKK

SEQ ID NO: 21 - protein, 7 aa, Artificial Sequence
  artificial peptide
RRKRRKK

SEQ ID NO: 22 - protein, 12 aa, Artificial Sequence
  artificial peptide
RRKRRKKRRKRR

SEQ ID NO: 23 - protein, 7 aa, Artificial Sequence
  artificial peptide
RKXRKKK

SEQ ID NO: 24 - protein, 7 aa, Artificial Sequence
  artificial peptide
RKXRKKK

SEQ ID NO: 25 - protein, 12 aa, Artificial Sequence
  artificial peptide
RKXRKKKKRXKR

SEQ ID NO: 26 - protein, 9 aa, Artificial Sequence
  artificial peptide
RKXRKKKKK

SEQ ID NO: 27 - protein, 9 aa, Artificial Sequence
  artificial peptide
XRGRKXXRX

SEQ ID NO: 28 - protein, 10 aa, Artificial Sequence
  C2-V2D or C2-V2 dimer
RGRKVVRRKK

SEQ ID NO: 29 - protein, 10 aa, Artificial Sequence
  C4-V2D or C4-V2 dimer
RGRKVVRRKK

SEQ ID NO: 30 - protein, 10 aa, Artificial Sequence
  C6-V2D or C6-V2 dimer
RGRKVVRRKK

SEQ ID NO: 31 - protein, 10 aa, Artificial Sequence
  C8-V2D or C8-V2-dimer
RGRKVVRRKK

SEQ ID NO: 32 - protein, 10 aa, Artificial Sequence
  C10-V2D or C10-V2-dimer
RGRKVVRRKK

SEQ ID NO: 33 - protein, 10 aa, Artificial Sequence
  C12-V2D or C12-V2-dimer
RGRKVVRRKK

SEQ ID NO: 34 - protein, 10 aa, Artificial Sequence
  C14-V2D or C14-V2-dimer
RGRKVVRRKK

SEQ ID NO: 35 - protein, 8 aa, Artificial Sequence
  C16-V2D or C16-V2-dimer
RGRKVVKK

SEQ ID NO: 36 - protein, 10 aa, Artificial Sequence
  C2-G2D or C2-G2-dimer
RGRKGGRRKK

SEQ ID NO: 37 - protein, 10 aa, Artificial Sequence
  C4-G2D or C4-G2-dimer
RGRKGGRRKK

SEQ ID NO: 38 - protein, 10 aa, Artificial Sequence
  C6-G2D or C6-G2-dimer
RGRKGGRRKK

SEQ ID NO: 39 - protein, 10 aa, Artificial Sequence
  C8-G2D or C8-G2-dimer
RGRKGGRRKK

SEQ ID NO: 40 - protein, 10 aa, Artificial Sequence
  C10-G2D or C10-G2-dimer
RGRKGGRRKK

SEQ ID NO: 41 - protein, 10 aa, Artificial Sequence
  C12-G2D or C12-G2-dimer
RGRKGGRRKK

SEQ ID NO: 42 - protein, 10 aa, Artificial Sequence
  C14-G2D or C14-G2-dimer
RGRKGGRRKK

SEQ ID NO: 43 - protein, 10 aa, Artificial Sequence
  C16-G2D or C16-G2-dimer
RGRKGGRRKK

SEQ ID NO: 44 - protein, 10 aa, Artificial Sequence
  artificial peptide
RARKGGRRKK

SEQ ID NO: 45 - protein, 9 aa, Artificial Sequence
  artificial peptide
RARKGRRKK

SEQ ID NO: 46 - protein, 9 aa, Artificial Sequence
  artificial peptide
RARKARRKK

SEQ ID NO: 47 - protein, 10 aa, Artificial Sequence
  V2D or V2 dimer or B2088
RGRKVVRRKK

(19) United States
(12) Patent Application Publication
(10) Pub. No.: US 2015/0231199 A1
(43) Pub. Date: Aug. 20, 2015

(54) PEPTIDES AND THEIR USES

(71) Applicants: AGENCY FOR SCIENCE, TECHNOLOGY AND RESEARCH, Singapore (SG); SINGAPORE HEALTH SERVICES PTE LTD, Singapore (SG)

(72) Inventors: Verma Chandra Shekhar, Singapore (SG); Jianguo Li, Singapore (SG); Lakshminarayanan Rajamani, Singapore (SG); Roger Wilmer Beuerman, Singapore (SG); Shouping Liu, Singapore (SG); Jun Jie Koh, Singapore (SG)

(21) Appl. No.: 14/426,695

(22) PCT Filed: Sep. 9, 2013
(86) PCT No.: PCT/SG2013/000392

§ 371 Date: Mar. 6, 2015

(30) Foreign Application Priority Data
Sep. 7, 2012 (SG) 201206671-8

(51) Int. Cl. A61K 38/16; A61K 45/06; A61K 38/10; C07K 7/08; C07K 14/00

(52) U.S. Cl. CPC A61K 38/16; C07K 7/08; C07K 14/00; A61K 38/10; A61K 45/06

(57) ABSTRACT

Disclosed are antimicrobial peptides. Also disclosed are methods of treating bacterial infection and fungal infection and a method of removing biofilm. Also disclosed is the use of these peptides.

### CROSS-REFERENCE TO RELATED APPLICATIONS

This application claims the benefit of priority of Singapore patent application No. 201206671-8, filed 7 Sep. 2012, the contents of it being hereby incorporated by reference in its entirety for all purposes.

### FIELD OF THE INVENTION

The present invention generally relates to the field of molecular biology and biochemistry and in particular to antimicrobial peptides and methods for their use and uses thereof.

### BACKGROUND OF THE INVENTION

Antimicrobial agents are agents that are used to inhibit the growth of or kill microbes. Various antimicrobials such as antibiotics or antibacterials, antifungals, antivirals or anti-parasites are known in the art. The most commonly known antimicrobials is antibiotics, which can be applied to various applications in medical and non-medical settings. However, due to the indiscriminate use of antibiotics in all walks of life, antibiotic resistant bacteria are on the rise. Resistance of a microorganism, such as a bacterium to an antibiotic can range from substantially greater tolerance or reduced susceptibility to completely unaffected by the antibiotics. When a microorganism cannot be controlled or killed by antibiotics or antibacterial agents, the microorganism is able to survive, multiply and cause disease or damages to the hosts despite being in the presence of the antibiotic. Such antibiotic resistant microorganism has become a significant public health threat.

In view of the above, there is a need to provide an alternative peptide that can be used against microorganism.

### SUMMARY OF THE INVENTION

In one aspect, there is provided a peptide comprising Formula I (SEQ ID NO: 1):

[(R)ₐ(X¹)_{b}(X²)_{c}(X³)ₐ(X⁴)_{b}]ₙ.

In one example, X¹, X² and X⁴ are independently of each other selected from the group consisting of K, R, G and A; X³ is K, R, L, V, I, G or A. In one example, a and b is independently selected to be an integer from 1 to 10. In one example, c is an integer selected from 0 to 5. In one example, n is at least one.

In another aspect, there is provided a peptide comprising Formula VIII (SEQ ID NO: 27):

X⁷[RGRK(X⁸)(X⁹)(R)_{f}]ₙ(X¹⁰)_{g}.

In one example, X⁷ is a lipid group. In one example, X⁸ and X⁹ are independently of each other, selected from the group consisting of Valine (V), Isoleucine (I), Leucine (L), Alanine (A) and Glycine (G). In one example, X¹⁰ is selected from the group consisting of Lysine (K) and Arginine (R). In one example, e and f are independently of each other an integer selected from 0 to 2. In one example, n is at least one.

In another aspect, there is provided a peptide as described herein for use as a medicament.

In another aspect, there is provided a composition comprising a peptide as described herein.

In another aspect, there is provided a method of treating a bacterial infection or removing bacteria. The method comprises the administration of a pharmaceutically effective amount of a peptide as described herein.

In another aspect, there is provided a method of neutralizing endotoxins. The method comprising administration of a pharmaceutically effective amount of a peptide as described herein.

In another aspect, there is provided a method of treating a fungal infection or infestation, or removing fungus. The method comprising administration of a pharmaceutically effective amount of a peptide as described herein.

In another aspect, there is provided method of removing a biofilm. The method comprising administration of an effective amount of a peptide as described herein.

In another aspect, there is provided a use of a peptide as described herein in the manufacture of a medicament for treating a bacterial infection, or removing bacteria, or neutralizing endotoxins, or treating a fungal infection of infestation, or removing fungus.

In another aspect, there is provided a kit comprising the peptide as described herein and its instructions thereof.

### BRIEF DESCRIPTION OF THE DRAWINGS

The invention will be better understood with reference to the detailed description when considered in conjunction with the non-limiting examples and the accompanying drawings, in which:

FIG. 1 shows a dot plot of in vitro time-kill kinetic assay for C8V2D against P. aeruginosa. Thus, FIG. 1 shows that an exemplary peptide of the present invention is effective in killing microbes.

FIG. 2 shows photographical images of cornea after topical toxicity test. In topical toxicity test, cornea clarity was checked by slit lamp microscopy with follow up every day for four days after the application of V2D and C8V2D.

FIG. 3 (A) shows a dot-plot illustrating the effect of the length of lipid modification of a peptide to its ability to effectively neutralize lipopolysaccharides from E. coli. (B) shows a dot-plot illustrating the effect of the length of lipid modification of a peptide to its ability to effectively neutralize lipopolysaccharides from P. aeruginosa.

FIG. 4 shows a curved graph showing the displacement of bodipy TR cadaverine (BC) fluorescent probe from lipopolysaccharide by the peptide and lipid-modified peptides of the present disclosure. FIG. 4 shows effective displacement of BC by the lipid-modified peptides of the present disclosure. Thus, illustrating that the lipid-modified peptides of the present disclosure are effective in neutralising lipopolysaccharides.

FIG. 5 shows a bar graph of results obtained from the study into the effect of peptide and lipid-modified peptides of the present disclosure on the permeabilisation of lipopolysaccharides. Inner left bar represents 1.5625 (μg/ml), with increasing concentration towards the right hand side of the x-axis. FIG. 5 shows the lipid-modified peptides of the present disclosure are effective in inducing lipopolysaccharides permeabilisation.

FIG. 6 shows the effect of the lipid-modified peptides of the present disclosure in changing the membrane potential of S. aureus DM4001 (A) or E. coli ATCC8739 (B). Change in membrane potential in the bacteria is observed in the change of fluorescence intensity of DiSC3-5, which is presented in count per second (c.p.s.). Thus, FIG. 6 shows that the lipid-modified peptides of the present disclosure are effective in causing changes in the membrane potential of both S. aureus and E. coli.

FIG. 7 shows the degree of permeabilisation of the inner membrane of S. aureus by the lipid-modified peptides of the present disclosure as observed by backlight assay. FIG. 7 shows that the C16-V2D lipid-modified peptide of the present disclosure to be effective permeabilising the inner membrane of S. aureus.

FIG. 8 shows the results of the study into the effect of the lipid-modified peptides of the present disclosure in causing calcein leakage from liposome which mimics bacterial membrane (A) or red blood cell (B). Thus, FIG. 8 shows the lipid-modified peptides of the present disclosure are selectively causing membrane leakage to bacterial membranes and not towards mammalian red blood cells.

FIG. 9 shows the design of the lipid-modified peptides or lipopeptides of the present disclosure. It is believed that the lipid-modified peptides or lipopeptides of the present disclosure binds with lipopolysaccharides via electrostatic and hydrophobic interaction. (A) shows the structure of an example of the lipid-modified peptides of the present disclosure. (B) shows the structure of lipopolysaccharides. (C) shows the electrostatic or hydrophobic interaction between lipid-modified peptides of the present disclosure and lipopolysaccharides.

FIG. 10 shows the results of in vivo testing of the peptide of the present disclosure in combination with a second therapeutic agent (gatifloxacin and B2088) on mouse cornea infected with Pseudomonas aeruginosa (ATCC 9027). FIG. 10 shows the combination of the peptide of the present invention and an antibiotic lead to efficient inhibition of Pseudomonas aeruginosa.

FIG. 11 shows the results of in vivo testing of peptide of the present disclosure in combination with a second therapeutic agent (gatifloxacin and B2088/99) on mouse cornea infected with Pseudomonas aeruginosa (ATCC 9027). FIG. 11 shows the combination of the peptide of the present invention and an antibiotic provides synergistic effect that leads to the most effective inhibition of Pseudomonas aeruginosa.

FIG. 12 shows the bactericidal properties of B2088 (i.e. V2D) and B2088_{—}99 (i.e. G2 dimer) against (a) P. aeruginosa ATCC 9027 and (B) P. aeruginosa ATCC 27853 strains. Note that the effective dose of peptide that reduces the viability of bacteria cells by 50% (ED₅₀) is two times lower for B2088_{—}99 (i.e. G2 dimer) than B2088 (i.e. V2 dimer). Thus, FIG. 12 shows effective killing of bacteria by the peptide of the present disclosure. [

FIG. 13 shows time-kill kinetics of B2088 (i.e. V2 dimer) and B2088_{—}99 (i.e. G2 dimer) against P. aeruginosa. Note that B2088_{—}99 (i.e. G2 dimer) displayed faster kill kinetics against both the Pseudomonas strains at 1× and 2×MIC. Thus, FIG. 13 shows that the peptide of the present disclosure causes faster bacterial killing than controls.

FIG. 14 shows the results of outer membrane (OM) permeability assay of B2088 (i.e. V2 dimer) and B2088_{—}99 (i.e. G2 dimer). The peptide concentration required to cause 50% increase in the NPN fluorescence intensity (PC₅₀) was measured. PC50 for B2088_{—}99 (i.e. G2 dimer) was higher than B2088 (i.e. V2 dimer), indicating that B2088 (i.e. V2D) has superior permeabilising effect as compared to B2088_{—}99 (i.e. G2 dimer).

FIG. 15 shows the interaction of B2088 (i.e. V2 dimer) and B2088_{—}99 (i.e. G2 dimer) with (A) lipopolysaccharide (LPS) and (B) Lipid A. The Bodipy displacement assay suggested that B2088 peptide binds 2 times more strongly to LPS and >10 times more strongly to Lipid A than B2088_{—}99 (i.e. G2 dimer). (C) shows the results of competitive inhibition assay showing the effect of exogenous addition of LPS on the inhibitory activity of peptides. The results suggest that B2088_{—}99 (i.e. G2 dimer) may not have superior LPS neutralization effect as B2088 (i.e. V2 dimer). These results further confirm that peptide B2088_{—}99 (i.e. G2 dimer), as compared to B2088, weakly binds to LPS. (D) shows the effect of Mg²⁺ ion on the minimum inhibitory concentration value (MIC) of B2088 (i.e. V2 dimer) and B2088_{—}99 (i.e. G2 dimer). Mg²⁺ stabilizes the outer membrane of Gram-negative bacteria and antagonizes the permeability of cationic agents. FIG. 15D shows B2088 (i.e. V2 dimer) binding to Lipid A and LPS is strongly dependent on Mg²⁺ concentration.

### BRIEF DESCRIPTION OF THE TABLES

Table 1A shows comparison of antimicrobial activities as presented by minimal inhibitory values (MIC) of lipid modified V2-dimer (C2-C14 V2D) and V2-dimer (V2D). Table 1A shows significant improvement in the inhibition of microorganisms Pseudomonas aeruginosa, Methicillin-resistant Staphylococcus aureus (MRSA), Klebsiella pneumoniae, Escherichia coli and Staphylococcus aureus by lipid modified C8-V2-dimer as compared to non-modified V2 dimer peptide.

Table 1B shows comparison of minimal inhibitory values of lipid modified G2-dimer (C2-C14 G2D) and G2-dimer (G2D). Table 1B shows significant improvement in the inhibition of microorganisms Pseudomonas aeruginosa, Staphylococcus aureus, Methicillin-resistant Staphylococcus aureus (MRSA) and K. Pneumoniae by lipid modified C8-G2-dimer and C10-G2-dimer as compared to non-modified G2 dimer peptide.

Table 2A shows a comparison of the antibacterial activity of V2D, C8-V2D and C10-V2D against a panel of Methicillin-sensitive Staphylococcus aureus (MSSA) and Methicillin-resistant Staphylococcus aureus (MRSA) strains. Table 2A shows that of the 16 strains of Staphylococcus aureus tested, C8-V2D dimer provided two fold improvements toward eight strains and 4 fold improvements toward one strain. Thus, providing an overall improvement of 56.3%. C10-V2D dimer provided two fold improvements toward ten strains. Thus, providing an overall improvement of 62.5%.

Table 2B shows antibacterial activity of V2D, C8-V2D and C10-V2D against a panel of Pseudomonas aeruginosa. Table 2B shows that of the 10 strains of Pseudomonas aeruginosa tested, C8-V2D dimer provided two fold improvements toward seven strains and 4 fold improvements toward one strain. Thus, providing an overall improvement of 80%. C10-V2D dimer provided two fold improvements toward seven strains and 4 fold improvements toward one strain. Thus, providing an overall improvement of 80%.

Table 3A shows the hemolytic activity of peptide and lipid-modified peptides of the present disclosure. Table 3A demonstrates that the lipid-modified peptides of the present disclosure advantageously do not cause hemolysis.

Table 3B shows the results of in vitro toxicity study of C8V2D compared to V2D. Table 3B shows an example of the lipid-modified peptides of the present disclosure to be non-toxic in vitro.

Table 3C shows the safety concentration of C8V2D in in vivo topical and acute toxicity tests. Table 3C shows that when applied topically, the peptide of the present disclosure is tolerated at more than 3 mg/kg; when applied intravenously, the peptide of the present disclosure is tolerated at about 6.25 mg/kg; when applied intraperitoneally, the peptide of the present disclosure is tolerated at about 100 mg/kg.

Table 4A shows the effective concentration of V2D and lipid modified V2D to neutralize 50% of lipopolysaccharides (LPS) from E. coli. Table 4A shows the lipid-modified peptides of the present disclosure are effective in neutralizing lipopolysaccharides from E. coli.

Table 4B shows the effective concentration of V2D and lipid modified V2D to neutralize 50% of lipopolysaccharides (LPS) from Pseudomonas aeruginosa. Table 4B shows the lipid-modified peptides of the present disclosure are effective in neturalizing lipopolysaccharides from Pseudomonas.

Table 5 shows fractional inhibition concentration index of V2D, C6V2D and C8V2D with five different antibiotics against bacteria. Table 5 shows C6-V2 dimer of the lipid-modified peptide of the present disclosure had better synergism effect in three out of five antibiotics tested against Pseudomonas aeruginosa; As compared to C6-V2 dimer, C8-V2 dimer has weaker effect against Pseudomonas aeruginosa. In contrast, C8-V2 dimer of the lipid-modified peptide of the present disclosure has much better synergism effect than non-lipid-modified peptide when tested against Escherichia coli.

Table 6 shows the minimum inhibitory concentration (MIC) of B2088 (i.e. V2D) and B2088_{—}99 (i.e. G2D).

Table 7 shows the bactericidal properties of B2088 (i.e. V2D) and B2088_{—}99 (i.e. G2D) as measured by ED50 (effective dose to kill 50% of bacterial cells).

Table 8 shows the synergism between B2088 (i.e. V2D) and B2088_{—}99 (i.e. G2D) with various classes of antibiotics. The multidrug resistant strains P. aeruginosa DR4877 was used for the experiments. Fractional inhibitory concentration (FIC) index was used to characterize the synergistic effect of the combination of the peptides of the present disclosure with an antibiotic. FIC index<0.5 synergistic; additivity, 0.5<FIC index>1.0; indifference, 1<FIC index<4; FIC index>4, antagonism.

### DETAILED DESCRIPTION OF THE PRESENT INVENTION

Antimicrobial peptides are normally characterized as small cationic hydrophobic peptides. As commonly known in the art, the inclusion of hydrophobic moieties was considered in the art to be critical for antimicrobial action against the membrane of bacteria. An example of such antimicrobial peptide is provided herein. That is, in one aspect, there is provided a peptide comprising Formula I (SEQ ID NO: 1):

[(R)ₐ(X¹)_{b}(X²)_{c}(X³)ₐ(X⁴)_{b}]ₙ,

wherein X¹, X² and X⁴ are independently of each other selected from the group consisting of lysine (K), arginine (R), glycine (G) and alanine (A); and X³ is lysine (K), arginine (R), leucine (L), valine (V), isoleucine (I), glycine (G) or alanine (A).

At the same time, contrary to popular believe, the inventors of the present disclosure surprisingly found that enhanced antimicrobial action can be observed in peptides that do not include hydrophobic amino acids. Thus, the present disclosure also provides for peptides with effective antimicrobial actions that are enhanced by the omission of hydrophobic amino acids. For example, X¹, X², X³ and X⁴ of Formula I (SEQ ID NO: 1) may be independently of each other selected from the group consisting of non-hydrophobic amino acids. In one example, X¹, X², X³ and X⁴ may be the same or different from one another. In one example, X¹, X², X³ and X⁴ may not be hydrophobic amino acid. In one example, X¹, X², X³ and X⁴ may not be valine (V), isoleucine (I), leucine (L), methionine (M), phenylalanine (F), tyrosine (Y) or tryptophane (W). In one example, X¹, X², X³ and X⁴ may be neutral amino acids. In one example, X¹, X², X³ and X⁴ may be cationic amino acids. In one example, X¹, X², X³ and X⁴ may be independently of each other an amino acid including, but not limited to arginine (R), histidine (H), lysine (K), aspartic acid (D), glutamic acid (E), serine (S), threonine (T), asparagine (N), glutamine (Q), cysteine (C), glycine (G), proline (P) or analine (A). In one example, X¹, X², X³ and X⁴ may be lysine (K), arginine (R), glycine (G) or alanine (A). In one example, X¹ may be lysine (K), arginine (R), glycine (G) or alanine (A). In one example, X² may be lysine (K), arginine (R), glycine (G) or alanine (A). In one example, X³ may be lysine (K), arginine (R), glycine (G) or alanine (A). In one example, X⁴ may be lysine (K), arginine (R), glycine (G) or alanine (A). X¹, X², X³ and X⁴ may be any combination of the aforementioned amino acids. That is, in one example, X¹ may be a lysine (K), X² may be a glycine (G) or alanine (A), X³ may be an arginine (R) and X⁴ may be a lysine (K). In yet another example, X¹ may be a glycine (G) or alanine (A), X² may be a lysine (K), X³ may be a glycine (G) or alanine (A) and X⁴ may be an arginine (R).

In one, example, when n is at least 2, each of the peptide sequence is linked to at least two lysine (K) residues. As used herein, “linked” refers to when two sequences of a peptide are coupled or connected to one other in a manner which permits each peptide branch to move freely of each other. In order to be “linked” it is necessary that two sequences be immediately adjacent to one another. In one example, a plurality of monomers of the peptide as described herein is linked by the lysine (K) residues via covalent bonds.

As used herein, the term “peptide” refers to an isolated peptide. Additionally, as used herein, the term “amino acid” includes naturally and non-naturally occurring L- and D-amino acids, peptidomimetic amino acids and non-standard amino acids that are not made by a standard machinery or are only found in proteins after post-translational modification or as metabolic intermediates.

In one example, the lysine (K) linkage is at the C-terminal end. The term “C-terminal end” is used herein in accordance to its definition as commonly known in the art, that is, can be used interchangeably with any of the following terminologies such as the carboxyl-terminus, carboxy-terminus, C-terminal tail, C-terminus or COOH-terminus, which refer to the end of an amino acid chain, terminated by a free carboxyl group (—COOH). As written herein, the peptides as described herein are presented as C-terminal end on the right and N-terminal end on the left.

When the peptide of the present disclosure is provided as a branched peptide that is linked by a lysine (K) linkage, for example when n is 2, the peptide is a dimer and may have the structure: [(R)ₐ(X¹)_{b}(X²)_{c}(X³)ₐ(X⁴)_{b}]₂KK or [(R)ₐ(X¹)_{b}(X²)_{c}(X³)ₐ(X⁴)_{b}]—K—K[(X⁴)_{b}(X³)ₐ(X²)_{c}(X¹)_{b}(R)ₐ].

On the other hand, when n is 3, the peptide is a trimer and may have the structure: [(R)ₐ(X¹)_{b}(X²)_{c}(X³)ₐ(X⁴)_{b}]₃¹K₂K

In one example, when n is 4, the peptide is a tetramer and may have the structure [(R)ₐ (X¹)_{b}(X²)_{c}(X³)ₐ(X⁴)_{b}]₄K₃K.

In one example, X² may be a lysine (K) and X⁴ may be an arginine (R). In such example, the peptide may comprise Formula II (SEQ ID NO: 2):

[(R)ₐ(X)_{c}(K)_{b}(X)_{c}(R)ₐ(X)_{c}(K)_{b}]ₙ(K)ₙK.

In one example, X of Formula II (SEQ ID NO: 2) may be glycine (G) or alanine (A). In one example, X of Formula II (SEQ ID NO: 2) may be glycine (G). In one example, X of Formula II (SEQ ID NO: 2) may be alanine (A).

In one example, a and b may be independently selected from an integer from 1 to 10. In one example, a and b may be the same or different from one another. In one example, a may be 1, 2, 3, 4, 5, 6, 7, 8, 9 or 10. In one example, b may be 1, 2, 3, 4, 5, 6, 7, 8, 9 or 10. Thus, in one example, a may be 1 and b may be 1; a may be 1 and b may be 2; a may be 1 and b may be 3; a may be 1 and b may be 4; a may be 1 and b may be 5; a may be 1 and b may be 6; a may be 1 and b may be 7; a may be 1 and b may be 8; a may be 1 and b may be 9; a may be 1 and b may be 10; a may be 2 and b may be 1; a may be 3 and b may be 1; a may be 4 and b may be 1; a may be 5 and b may be 1; a may be 6 and b may be 1; a may be 7 and b may be 1; a may be 8 and b may be 1; a may be 9 and b may be 1; a may be 10 and b may be 1; a may be 2 and b may be 2; a may be 2 and b may be 3; a may be 2 and b may be 4; and any combination thereof.

In one example, c may be an integer selected from 0 to 5. In one example, c may be 0, 1, 2, 3, 4 or 5.

In one example, n may to at least one, may be at least two, may be at least three or four. In one example, n may be an integer selected from 1 to 8. Thus, n may be 1, 2, 3, 4, 5, 6, 7 or 8. In another example, n may not be an integer selected from 1 to 4. Thus, n may be 1, 1.5, 2, 2.5, 3, 3.5, 4, 4.5, 5, 5.5, 6, 6.5, 7, 7.5 or 8.

In one example, the peptide of Formula II (SEQ ID NO: 2) may include, but is not limited to [(R)ₐ(X)_{c}(K)_{b}(X)_{c}(R)ₐ(X)_{c}(K)_{b}]₂KK (SEQ ID NO: 3, which is a dimer), [(R)ₐ(X)_{c}(K)_{b}(X)_{c}(R)ₐ(X)_{c}(K)_{b}]₂KK[(K)_{b}(X)_{c}(R)ₐ(X)_{c}(K)_{b}(X)_{c}(R)ₐ] (SEQ ID NO: 4, which is a trimer), and [(R)ₐ(X)_{c}(K)_{b}(X)_{c}(R)ₐ(X)_{c}(K)_{b}]₄(K)₃K (SEQ ID NO: 5, which is a tetramer).

In one example, when X³ is arginine (R) and X⁴ is lysine (K), the peptide may comprise Formula III (SEQ ID NO: 6):

[R(X⁵)_{d}RK(X⁶)ₑRR]ₙ(K)ₙ₋₁K.

In one example, X⁵ and X⁶ may be the same or different from one another. In one example, X⁵ and X⁶ are independently of each other an amino acid including, but is not limited to arginine (R), histidine (H), lysine (K), aspartic acid (D), glutamic acid (E), serine (S), threonine (T), asparagine (N), glutamine (Q), cysteine (C), glycine (G), proline (P) or analine (A). In one example, X⁵ and X⁶ may be glycine (G), alanine (A) or arginine (R). In one example, X⁵ may be glycine (G) and X⁶ may be glycine (G). In one example, X⁵ may be alanine (A) and X⁶ may be glycine (G). In one example, X⁵ may be arginine (R) and X⁶ may be glycine (G). In one example, X⁵ may be glycine (G) and X⁶ may be alanine (A). In one example, X⁵ may be alanine (A) and X⁶ may be alanine (A). In one example, X⁵ may be arginine (R) and X⁶ may be alanine (A). In one example, X⁵ may be glycine (G) and X⁶ may be arginine (R). In one example, X⁵ may be alanine (A) and X⁶ may be arginine (R). In one example, X⁵ may be arginine (R) and X⁶ may be arginine (R).

In one example, d and e may be independently from each other an integer selected from 0 to 2. In one example, d or e may be 0, 1 or 2. In one example, d may be 0, 1 or 2. In one example, e may be 0, 1 or 2. Thus, d may be 0 and e may be 0; d may be 0 and e may be 1; d may be 0 and e may be 2; d may be 1 and e may be 0; d may be 1 and e may be 1; d may be 1 and e may be 2; d may be 2 and e may be 0; d may be 2 and e may be 1; or d may be 2 and e may be 2.

In one example, where X⁵ is alanine (A) and d is one, Formula III (SEQ ID NO: 6) may comprise Formula IV (SEQ ID NO: 7):

[RARK(X⁶)ₑRR]ₙ(K)ₙ₋₁K.

In one example, e may be an integer selected from 0 to 2. In one example, e may be 0, 1 or 2.

In one example, n may be at least one, may be at least two, may be at least three or may be four. In one example, n may be an integer. In one example, when n is an integer, n may be 1, 2, 3, 4, 5, 6, 7 or 8. In one example, n may not be an integer. Thus, n may be 1, 1.5, 2, 2.5, 3, 3.5, 4, 4.5, 5, 5.5, 6, 6.5, 7, 7.5 or 8.

In one example, X⁶ may be glycine (G) or alanine (A). Thus, the peptide of Formula IV may include, but is not limited to (RARKGGRR)₂KK (SEQ ID NO: 44), (RARKGRR)₂KK (SEQ ID NO: 45), (RARKARR)₂KK (SEQ ID NO: 46), (RARKAARR)₂KK (SEQ ID NO: 8) and (RARKRR)₂KK (SEQ ID NO: 9).

In one example, where X⁵ is glycine (G) and d is 1, Formula III (SEQ ID NO: 6) may comprise Formula V (SEQ ID NO: 10):

[RGRK(X⁶)ₑRR]ₙ(K)ₙ₋₁K.

In one example, X⁶ may be valine (V), glycine (G) or alanine (A). In one example, the peptide of Formula V may include, but is not limited to (RGRKGGRR)₂KK (SEQ ID NO: 11; or interchangeably used with the terms “B2088_{—}99”, “B2088/99”, “G2D” and “G2D-dimer”), (RGRKGGRR)₂KK (SEQ ID NO: 12), (RGRKGRR)₂KK (SEQ ID NO: 13), (RGRKRR)₂KK (SEQ ID NO: 14), (RGRKAARR)₂KK (SEQ ID NO: 15), (RGRKARR)₂KK (SEQ ID NO: 16), (RGRKGGRR)₂KKRRGGKRGR (SEQ ID NO: 17), (RGRKGRR)₂KKRRGKRGR (SEQ ID NO: 18), (RGRKRR)₂KKRRKRGR (SEQ ID NO: 19) and (RGRKVVRR)₂KK (SEQ ID NO: 47; or interchangeably used with the terms “B2088”, “V2D” and “V2D-dimer”).

In one example, when d and e are 0, Formula III (SEQ ID NO: 6) may comprise Formula VI (SEQ ID NO: 20):

[RRKRR]ₙ(K)ₙ₋₁K.

In one example, the peptide of Formula VI may include, but is not limited to (RRKRR)₂KK (SEQ ID NO: 21) and (RRKRR)₂KKRRKRR (SEQ ID NO: 22).

In one example, where X¹ is lysine (K), X³ is an arginine (R) and X⁴ is a lysine (K), the peptide of Formula II (SEQ ID NO: 2) may comprise Formula VII (SEQ ID NO: 23):

[(R)ₐ(K)_{b}(X)_{c}(R)ₐ(K)_{b}]ₙ(K)ₙ₋₁K.

In one example, X of Formula VII (SEQ ID NO: 23) may be a non-hydrophobic amino acid. In another word, X of Formula VII (SEQ ID NO: 23) may not be a hydrophobic amino acid. In one example, X of Formula VII (SEQ ID NO: 23) may be arginine (R), histidine (H), lysine (K), aspartic acid ° (D), glutamic acid (E), serine (S), threonine (T), asparagine (N), glutamine (Q), cysteine (C), glycine (G), proline (P) or alanine (A).

In one example, X of Formula VII (SEQ ID NO: 23) may be G or A. In one example, the peptide of Formula VII may include, but is not limited to [(R)ₐ(K)_{b}X_{c}(R)ₐ(K)_{b}]₂KK (SEQ ID NO: 24), [(R)ₐ(K)_{b}X_{c}(R)ₐ(K)_{b}]₂KK[(K)_{b}(R)ₐX_{c}(K)_{b}(R)ₐ] (SEQ ID NO: 25), and [(R)ₐ(K)_{b}X_{c}(R)ₐ(K)_{b}]₄K₃K (SEQ ID NO: 26).

In another aspect, there is provided a peptide comprising Formula VIII (SEQ ID NO: 27):

X⁷[RGRK(X⁸)(X⁹)(R)_{f}]ₙ(X¹⁰)_{g}.

In one example, X⁷ is a lipid group. In one example, X⁷ may be —RCONH, where R may include, but is not limited to alkyl optionally substituted by a hydroxyl group, a carbonyl group or an alkenyl group. For example, X⁷ may be CₘH₂ₘ₋₁—CONH, where m may be an integer selected from 1 to 25. In some examples of the peptides of the present disclosure, m may be 1, 2, 3, 4, 5, 6, 7, 8, 9, 10, 11, 12, 13, 14, 15, 16, 17, 18, 19, 20, 21, 22, 23, 24 or 25. When m is 25, the lipid group is cerotic acid modified. The lipid group may also include cis or trans-form of unsaturated fatty acid with single or multi-double bonds, which may be synthetic or derived from nature (such as fatty acid produced by microorganism).

For example, X⁷ may include, but is not limited to (CH₃)—CONH—, C₃H₇—CO—NH—, C₅H₁₁—CO—NH—, C₇H₁₅—CO—NH—, C₉H₁₉—CO—NH—, C₁₁H₂₃—CO— and C₁₅H₃₁—CO—NH—. In one example, the lipid group is covalently bonded to the peptide.

In the present disclosure, peptides that are attached to a lipid group may be described interchangeably with the term “lipopeptides” or “lipid-modified peptides”.

The lipid group of the lipid-modified peptide of the present disclosure may be coupled to the peptide by using methods known in the art, for example using the solid phase peptide synthesis (SPPS). In brief, the general principle of SPPS is using repeated cycles of coupling-wash-deprotection-wash process. That is, peptides are immobilized on solid-phase, for example small solid beads or resins, which may be insoluble and/or porous. After immobilisation, the peptides are treated with functional units. The free N-terminal amine of the immobilised peptide is then coupled to a single N-protected amino acid unit. This unit is then deprotected using appropriate reagent such as piperidine, revealing free N-terminal amine, which can be used to attach the next N-protected amino acid with free carboxylic group. The reaction mixture is filtered in each step, and the peptides immobilized on the beads or resins are retained during the filtration process, whereas liquid-phase reagents and by-products of synthesis are flushed away. To synthesize the lipid-modified peptides, instead of N-protected amino acid with free carboxylic acid, fatty acid with desired carbon length with free carboxylic acid is used in coupling process. The reagent used in coupling process is similar to those used in coupling two amino acids. Before the peptide is cleaved from the beads or resins by cleaving reagents such as trifluoroacetic acid (TFA), the growing peptides will remain covalently attached to the beads or resins. After cleaving, the peptides or lipid-modified peptides will be collected and purified using High-performance liquid chromatography (HPLC).

The peptides of the present disclosure may be coupled to a fatty acid, which has a COOH group at one end. For example, the peptide may be couple to a palmitic acid

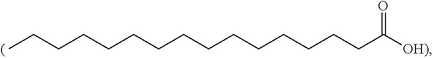

which is coupled to the NH₂ group of the N-terminal of the peptides of the present disclosure.

The lipid group of the peptides of the present disclosure may be coupled using appropriate peptide coupling agent such as, but is not limited to benzotriazol-1-yl-oxytripyrrolidinophosphonium hexafluorophosphate, N,N′-Dicyclohexylcarbodiimide and the like.

In one example, X⁸ and X⁹ may be independently of each other. In one example, X⁸ and X⁹ may be the same or different from one another. In one example, X⁸ and X⁹ may be arginine (R), histidine (H), lysine (K), aspartic acid (D), glutamic acid (E), serine (S), threonine (T), asparagine (N), glutamine (Q), cysteine (C), glycine (G), proline (P), alanine (A), isoleucine (I), leucine (L) or valine (V). In one example, X⁸ and X⁹ may be valine (V), alanine (A), isoleucine (I), leucine (L) or glycine (G). In another example, X⁸ and X⁹ may be valine (V) or glycine (G).

In one example, X¹⁰ may be arginine (R), histidine (H), lysine (K), aspartic acid (D), glutamic acid (E), serine (S), threonine (T), asparagine (N), glutamine (Q), cysteine (C), glycine (G), proline (P), alanine (A) or valine (V). In one example, X¹⁰ may be a cationic amino acid such as histidine (H), lysine (K) or arginine (R). In one example, X¹⁰ may be lysine (K) or arginine (R).

In one example, f and g may be independently from each other an integer selected from 0 to 2. In one example, f or g may be 0, 1 or 2. In one example, f may be 0, 1 or 2. In one example, g may be 0, 1 or 2. Thus, f may be 0 and g may be 0; f may be 0 and g may be 1; f may be 0 and g may be 2; f may be 1 and g may be 0; f may be 1 and g may be 1; f may be 1 and g may be 2; f may be 2 and g may be 0; f may be 2 and g may be 1; or f may be 2 and g may be 2.

In one example, n is at least one. In one example, n may be 1, 2, 3, 4, 5, 6, 7 or 8.

In one example, where X⁸ and X⁹ are Valine (V), Formula VIII (SEQ ID NO: 27) may be referred to as lipid modified V2 dimers or V2D or B2088 (i.e. (RGRKVVRR)₂KK) SEQ ID NO: 47). In one example, the peptides may include, but is not limited to (CH₃—CO—NH—RGRKVVRR)₂KK (SEQ ID NO: 28 or C2-V2-dimer), (C₃H₇—CO—NH—RGRKVVRR)₂KK (SEQ ID NO: 29 or C4-V2-dimer), (C₅H₁₁—CO—NH—RGRKVVRR)₂KK (SEQ ID NO: 30 or C6-V2-dimer), (C₇H₁₅—CO—NH—RGRKVVRR)₂KK (SEQ ID NO: 31 or C8-V2-dimer), (C₉H₁₉—CO—NH—RGRKVVRR)₂KK (SEQ ID NO: 32 or C10-V2-dimer), (C₁₁H₂₃—CO—NH—RGRKVVRR)₂KK (SEQ ID NO: 33 or C12-V2-dimer), (C₁₃H₂₃—CO—NH—RGRKVVRR)₂KK (SEQ ID NO: 34 or C14-V2-dimer) and (C₁₅H₃₁—CO—NH—RGRKVV)₂KK (SEQ ID NO: 35 or C16-V2-dimer). In one example, the peptide may be (C₇H₁₅—CO—NH—RGRKVVRR)₂KK (SEQ ID NO: 31 or C8-V2-dimer) and (C₉H₁₉—CO—NH—RGRKVVRR)₂KK (SEQ ID NO: 32 or C10-V2-dimer). As shown in Tables 1A, 2A, 2B, 4A and 4B, as compared to non-lipid modified peptide, these lipid-modified peptides have improved antimicrobial activities.

In one example, where X⁸ and X⁹ are glycine (G), Formula VIII (SEQ ID NO: 27) may be referred to as lipid modified G2 dimers or G2D or B2088_{—}99 or B2088/99 (i.e. (RGRKGGRR)₂KK) (SEQ ID NO: 11)). In one example, the peptide may include, but is not limited to (CH₃—CO—NH—RGRKGGRR)₂KK (SEQ ID NO: 36 or C2-G2-dimer), (C₃H₇—CO—NH—RGRKGGRR)₂KK (SEQ ID NO: 37 or C4-G2-dimer), (C₅H₁₁—CO—NH—RGRKGGRR)₂KK (SEQ ID NO: 38 or C6-G2-dimer), (C₇H₁₅—CO—NH—RGRKGGRR)₂KK (SEQ ID NO: 39 or C8-G2-dimer), (C₉H₁₉—CO—NH—RGRKGGRR)₂KK (SEQ ID NO: 40 or C10-G2-dimer), (C₁₁H₂₃—CO—NH—RGRKGGRR)₂KK (SEQ ID NO: 41 or C12-G2-dimer), (C₁₃H₂₃—CO—NH—RGRKGGRR)₂KK (SEQ ID NO: 42 or C14-G2-dimer) and (C₁₅H₃₁—CO—NH—RGRKGGRR)₂RR (SEQ ID NO: 43 or C16-G2-dimer). In one example, the peptide may be (C₇H₁₅—CO—NH—RGRKGGRR)₂KK (SEQ ID NO: 39 or C8-G2-dimer) and (C₉H₁₉—CO—NH—RGRKGGRR)₂KK (SEQ ID NO:40 or C10-G2-dimer). As shown in Table 1B, as compared to non-lipid modified peptide, these lipid modified peptides have improved antimicrobial activities.

In one example, the peptide as described herein may be chemically modified. In one example, the chemical modification may include, but is not limited to amidation, acetylation, stapling, replacing at least one L-amino acid with a corresponding D-amino acid, introducing or replacing at least one amino acid with a non-natural amino acid and lipidation. As used herein, the term “lipidation” refers to modification that results in the covalent binding a lipid group to a peptide chain. Lipidation may include, but is not limited to N-Myristoylation, Palmitoylation, GPI-anchor addition, Prenylation, Lipidation of bacterial proteins (S-diacylglycerol) and other types of lipidation.

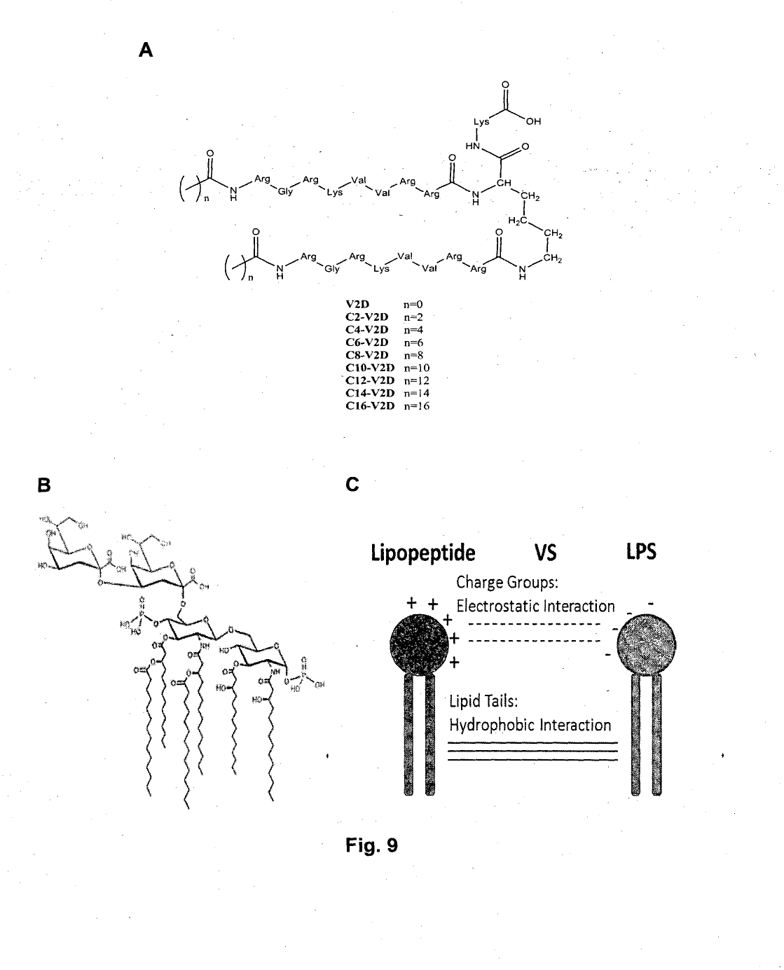

In one example, the peptide as described herein may further comprise a second therapeutic agent. The inventors of the present disclosure found a surprising synergistic effect when combining the peptide as described herein with a second therapeutic agent, such as an antibiotic. Without wishing to be bound by theory, the inventors believe that the lipid group of the peptides as described herein is important in sensitizing lipopolysaccharides. The peptides of the present disclosure are believed to neutralize lipopolysaccharides by effectively binding to lipopolysaccharides via both electrostatic and hydrophobic interactions (see FIG. 9C for an illustration of the interactions between a lipid-modified peptide of the present disclosure with lipopolysaccharide). The lipopolysaccharide is neutralized by disrupting the integrity of the lipopolysaccharides structures. As the lipopolysaccharides is the main barrier that protects Gram-negative bacteria from antimicrobial attack, neutralization of lipopolysaccharides causes Gram-negative bacteria to be more susceptible to other antimicrobials. Thus, the ability of disrupting lipopolysaccharides structural integrity advantageously allows the peptides of the present disclosure to synergize with other antimicrobials effectively. That is the combination of the peptide as described herein with a second therapeutic agent such as antibiotic, synergistically kills microbes. As used herein, the term “synergistic” refers to an effect that is greater than the sum of antimicrobial effect observed by the peptide of the present disclosure and a second therapeutic agent. That is, the combination of the peptide of the present disclosure with a second therapeutic agent provides for an antimicrobial effect that is greater than the sum of the individual effect of the peptide of the present disclosure and the second therapeutic agent.

A synergistic effect may be attained when the peptides of the present disclosure and the second therapeutic agent are: (1) co-formulated and administered or delivered simultaneously in a combined formulation; (2) delivered by alternation or in parallel as separate formulations; or (3) by some other regimen. When delivered in alternation therapy, a synergistic effect may be attained when the peptides of the present disclosure and the second therapeutic agent are administered or delivered sequentially, e.g., in separate tablets, pills or capsules, or by different injections in separate syringes. In general, during alternation therapy, an effective dosage of each peptides of the present disclosure and the second therapeutic agent is administered sequentially, i.e. serially. Exemplary results illustrating the surprising synergistic effect are provided in Examples 8 and 11 in Experimental Section below.

As used herein, the term “antimicrobial” refers to an agent, such as a peptide of the present disclosure, which is capable of eliminating, reducing or preventing diseases caused by the microbes. As used herein, the term “microbes” or “microorganism” is used in its broadest sense and is therefore not limited in scope to prokaryotic organisms. Rather, the term “microorganism” includes within its scope bacteria, archaea, yeast, fungi, protozoa and algae.

In another aspect, there is provided a method of treating a bacterial infection or removing bacteria. The method comprises the administration of a pharmaceutically effective amount of a peptide as described herein. The terms “treat,” “treatment,” and grammatical variants thereof, refer to both therapeutic treatment and prophylactic or preventative measures, wherein the object is to prevent or slow down (lessen) an undesired physiological condition, disorder or disease or obtain beneficial or desired clinical results. Such beneficial or desired clinical results include, but are not limited to, alleviation of symptoms; diminishment of extent of condition, disorder or disease; stabilized (i.e. not worsening) state of condition, disorder or disease; delay or slowing of condition, disorder or disease progression; amelioration of the condition, disorder or disease state, remission (whether partial or total), whether detectable or undetectable; or enhancement or improvement of condition, disorder or disease. Treatment includes eliciting a cellular response that is clinically significant, without excessive levels of side effects. Treatment also includes prolonging survival as compared to expected survival if not receiving treatment.

In one example, the bacteria may be Gram-positive or Gram-negative bacteria. Thus, bacteria may be of genus including, but not limited to Acetobacter, Acinetobacter, Actinomyces, Agrobacterium spp., Azorhizobium, Azotobacter, Anaplasma spp., Bacillus spp., Bacteroides spp., Bartonella spp., Bordetella spp., Borrelia, Brucella spp., Burkholderia spp., Calymmatobacterium, Campylobacter, Chlamydia spp., Chlamydophila spp., Clostridium spp., Corynebacterium spp., Coxiella, Ehrlichia, Enterobacter, Enterococcus spp., Escherichia, Francisella, Fusobacterium, Gardnerella, Haemophilus spp., Helicobacter, Klebsiella, Lactobacillus spp., Lactococcus, Legionella, Listeria, Methanobacterium extroquens, Microbacterium multiforme, Micrococcus luteus, Moraxella catarrhalis, Mycobacterium spp., Mycoplasma spp., Neisseria spp., Pasteurella spp., Peptostreptococcus, Porphyromonas, Pseudomonas, Rhizobium, Rickettsia spp., Rodhalimaea spp., Rothia, Salmonella spp., Serratia, Shigella, Staphylococcus spp., Stenotrophomonas, Streptococcus spp., Treponema spp., Vibrio spp., Wolbachia, and Yersinia spp. In one example, the bacterial infection may be caused by bacteria including, but are not limited to Acetobacter aurantius, Acinetobacter baumannii, Actinomyces Israelii, Agrobacterium radiobacter, Agrobacterium tumefaciens, Azorhizobium caulinodans, Azotobacter vinelandii, Anaplasma phagocytophilum, Anaplasma marginale, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacteroides fragilis, Bacteroides gingivalis, Bacteroides melaminogenicus (Prevotella melaminogenica), Bartonella henselae, Bartonella quintana, Bordetella bronchiseptica, Bordetella pertussis, Borrelia burgdorferi, Brucella abortus, Brucella melitensis, Brucella suis, Burkholderia mallei, Burkholderia pseudomallei, Burkholderia cepacia complex, Burkholderia cenocepacia, Calymmatobacterium granulomatis, Campylobacter coli, Campylobacter fetus, Campylobacter jejuni, Campylobacter pylori, Chlamydia trachomatis, Chlamydophila. (such as C. pneumoniae, Chlamydophila psittaci, Clostridium botulinum, Clostridium difficile, Clostridium perfringens, Clostridium tetani), Corynebacterium diphtheriae, Corynebacterium fusiforme, Coxiella bumetii, Ehrlichia chaffeensis, Enterobacter cloacae, Enterococcus avium, Enterococcus durans, Enterococcus faecalis, Enterococcus faecium, Enterococcus galllinarum, Enterococcus maloratus, Escherichia coli, Francisella tularensis, Fusobacterium nucleatum, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus acidophilus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Neisseria gonorrhoeae, Neisseria meningitidis, Pasteurella multocida, Pasteurella tularensis Peptostreptococcus, Porphyromonas gingivalis, Pseudomonas aeruginosa, Rhizobium Radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Staphylococcus aureus, Staphylococcus epidermidis, Stenotrophomonas maltophilia, Streptococcus agalactiae, Streptococcus. avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema pallidum, Treponema denticola, Vibrio cholerae, Vibrio comma, Vibrio parahaemolyticus, Vibrio vulnificus, Wolbachia, Yersinia enterocolitica, Yersinia pestis and Yersinia pseudotuberculosis.

In one example, the bacteria may be drug resistant. In one example, the bacterial infection may cause conditions such as, but are not limited to pneumonia, tuberculosis, meningitis, diarrhoeal diseases, formation of biofilm, sepsis, listeriosis, gastroenteritis, toxic shock syndrome, hemorrhagic colitis, hemolytic uremic syndrome, Lyme Disease, gastric and duodenal ulcers, human ehrlichiosis, pseudomembranous colitis, cholera, salmonellosis, cat scratch fever, necrotizing fasciitis (GAS), streptococcal toxic shock syndrome, nosocomial and community associated infections, atherosclerosis, sudden infant death syndrome (SIDS), wound infection, septicemia, gastrointestinal disease, hospital-acquired endocarditis and blood stream infections.

The lipopolysaccharide (LPS) endotoxin, which has potent immunostimulating properties, is an integral structural component in the outer membrane leaflet of Gram-negative bacteria. LPS is shed continuously during microbial cell growth and division and is liberated in large amounts during cell death, often as a result of antibiotics therapies against bacterial infections. Upon release into the bloodstream, LPS aggregates are dissociated by LPS-binding plasma proteins (LBPs) to form LPS-LBP complexes which stimulate the host monocytes and macrophages to secrete various cytokines (e.g. TNF-α, IL-6, IL-8) and pro-inflammatory mediators (e.g. NO and reactive oxygen species). This activation of the innate immune system triggers a cascade of exaggerated immune responses resulting in a serious clinical syndrome known as septic shock, which could rapidly precipitate in multiple organ failure or death if left untreated. Thus, there is a need to provide a method of neutralizing endotoxins. Accordingly, in another aspect, there is provided a method of neutralizing endotoxins. The method comprising administration of a pharmaceutically effective amount of a peptide as described herein.

In one example, the endotoxins may be bacterial endotoxins or fungal endotoxins. In one example, the endotoxins may be polysaccharides, lipoteichoic acid, lipopolysaccharide or lipooligosaccharide.

Another problem that bacteria may pose towards human is the formation of biofilms. The formation of biofilms is a significant problem that is implicated in a variety of settings both the medical field and the non-medical field. Biofilm formation occurs when microbial cells adhere to each other and are embedded in a matrix of extracellular polymeric substance (EPS) on a surface. The growth of microbes in such a protected environment that is enriched with biomacromolecules (e.g. polysaccharides, nucleic acids and proteins) and nutrients allow for enhanced microbial cross-talk and increased virulence. As biofilm may develop in any supporting environment, a method or composition that can remove or prevent biofilm formation is needed. Thus, there is a need to provide a method of removing or preventing biofilm formation.

In another aspect, there is provided a method of removing a biofilm. The method comprising administration of an effective amount of a peptide as described herein. In one example, the biofilm may occur on surfaces. The term “surface” or “surfaces” used herein, refer to any surface whether medical or industrial, that provides an interface between a fluid, such as a liquid or air, and a solid. The interface between fluid and solid can be intermittent, and can be caused by flowing or stagnant fluid, aerosols, or other means for air-borne fluid exposure. A surface refers, in some examples, to a plane whose mechanical structure is compatible with the adherence of bacteria or fungi. In the context of the peptides and methods described herein, the terminology “surface” encompasses the inner and outer aspects of various instruments and devices, both disposable and non-disposable, medical and non-medical. Examples of non-medical uses include the hull of a ship, dockyard, food processors, mixers, machines, containers, water tanks, water filtrations, purification systems, preservatives in food industries, personal care products such as shampoo, cream, moisturizer, hand sanitizer, soaps and the like. Examples of medical uses include the entire spectrum of medical devices. Such “surfaces” may include the inner and outer aspects of various instruments and devices, whether disposable or intended for repeated uses. Examples include the entire spectrum of articles adapted for medical use, including scalpels, needles, scissors and other devices used in invasive surgical, therapeutic or diagnostic procedures; implantable medical devices, including artificial blood vessels, catheters and other devices for the removal or delivery of fluids to patients, artificial hearts, artificial kidneys, orthopaedic pins, plates and implants; catheters and other tubes (including urological and biliary tubes, endotracheal tubes, peripherally insertable central venous catheters, dialysis catheters, long term tunneled central venous catheters, peripheral venous catheters, short term central venous catheters, arterial catheters, pulmonary catheters, Swan-Ganz catheters, urinary catheters, peritoneal catheters), urinary devices (including long term urinary devices, tissue bonding urinary devices, artificial urinary sphincters, urinary dilators), shunts (including ventricular or arterio-venous shunts); prostheses (including breast implants, penile prostheses, vascular grafting prostheses, heart valves, artificial joints, artificial larynxes, otological implants), vascular catheter ports, wound drain tubes, hydrocephalus shunts, pacemakers and implantable defibrillators, dental implants, filings, dentures and the like. Other examples will be readily apparent to practitioners in these arts. Surfaces found in the medical environment also include the inner and outer aspects of pieces of medical equipment, medical gear worn or carried by personnel in the health care setting. Such surfaces can include counter tops and fixtures in areas used for medical procedures or for preparing medical apparatus, tubes and canisters used in respiratory treatments, including the administration of oxygen, of solubilised drugs in nebulisers and of aesthetic agents. Also included are those surfaces intended as biological barriers to infectious organisms in medical settings, such as gloves, aprons and face-shields. Commonly used materials for biological barriers may be latex-based or non-latex based. An example for a non-latex based biological barrier material may include vinyl. Other such surfaces can include handles and cables for medical or dental equipment not intended to be sterile. Additionally, such surfaces can include those non-sterile external surfaces of tubes and other apparatus found in areas where blood or body fluids or other hazardous biomaterials are commonly encountered. In one example, the biofilm may be comprised on catheters and medical implants.

In another aspect, there is provided a method of treating a fungal infection or infestation, or removing fungus. The method comprising administration of a pharmaceutically effective amount of a peptide as described herein. As used herein, the term “fungi” (and derivatives thereof, such as “fungal infection”) includes, but is not limited to, references to organisms (or infections due to organisms) of the following genus Absidia, Ajellomyces, Arthroderma, Aspergillus, Blastomyces, Candida, Cladophialophora, Coccidioides, Cryptococcus, Cunninghamella, Epidermophyton, Exophiala, Filobasidiella, Fonsecaea, Fusarium, Geotrichum, Histoplasma, Hortaea, Issatschenkia, Madurella, Malassezia, Microsporum, Microsporidia, Mucor, Nectria, Paecilomyces, Paracoccidioides, Penicillium, Pichia, Pneumocystis, Pseudallescheria, Rhizopus, Rhodotorula, Scedosporium, Schizophyllum, Sporothrix, Trichophyton, and Trichosporon. In one example, the fungus may include, but is not limited to Absidia corymbifera, Ajellomyces capsulatus, Ajellomyces dermatitidis, Arthroderma benhamiae, Arthroderma fulvum, Arthroderma gypseum, Arthroderma incurvatum, Arthroderma otae and Arthroderma vanbreuseghemii, Aspergillus flavus, Aspergillus fumigatus and Aspergillus niger, Blastomyces dermatitidis, Candida albicans, Candida glabrata, Candida guilliermondii, Candida krusei, Candida parapsilosis, Candida tropicalis and Candida pelliculosa, Cladophialophora carrionii, Coccidioides immitis and Coccidioides posadasii, Cryptococcus neoformans, Cunninghamella Sp, Epidermophyton floccosum, Exophiala dermatitidis, Filobasidiella neoformans, Fonsecaea pedrosoi, Fusarium solani, Geotrichum candidum, Histoplasma capsulatum, Hortaea werneckii, Issatschenkia orientalis, Madurella grisae, Malassezia furfur, Malassezia globosa, Malassezia obtusa, Malassezia pachydermatis, Malassezia restricta, Malassezia slooffiae, Malassezia sympodialis, Microsporum canis, Microsporum fulvum, Microsporum gypseum, Microsporidia, Mucor circinelloides, Nectria haematococca, Paecilomyces variotii, Paracoccidioides brasiliensis, Penicillium marneffei, Pichia anomala, Pichia guilliermondii, Pneumocystis jiroveci, Pneumocystis carinii, Pseudallescheria boydii, Rhizopus oryzae, Rhodotorula rubra, Scedosporium apiospermum, Schizophyllum commune, Sporothnx schenckii, Trichophyton mentagrophytes, Trichophyton rubrum, Trichophyton verrucosum and Trichophyton violaceum, and Trichosporon asahii, Trichosporon cutaneum, Trichosporon inkin and Trichosporon mucoides.

In another aspect, there is provided a kit comprising the peptide as described herein and its instructions thereof.

In another aspect, there is provided a peptide as described herein for use as a medicament. In one example, the medicament may further comprise a second therapeutic agent.

In another aspect, there is provided a composition comprising a peptide as described herein. In one example, the composition may further comprise a second therapeutic agent.

In one example, the methods, uses or kits as described herein may further comprise a second therapeutic agent. In one example, the methods as described herein may further comprise the administration of a second therapeutic agent. In one example, the medicament as described herein may further comprise a second therapeutic agent. In one example, the medicament may be administered with a second therapeutic agent. In one example, the kits may further comprise a second therapeutic agent.

In one example, the second therapeutic agent may be administered separately or together with the peptide of the present disclosure. In one example, the second therapeutic agent may be a further or different antimicrobial agent. In one example, the antimicrobial agent may include, but is not limited to an antifungal, antiviral, antibacterial or an antibiotic and an anti-parasite. In one example, the antimicrobial is an antibiotic.

In one example, the antibiotic may include, but is not limited to Ampicillin, Bacampicillin, Carbenicillin Indanyl, Mezlocillin, Piperacillin, Ticarcillin, Amoxicillin-Clavulanic Acid, Ampicillin-Sulbactam, Benzylpenicillin, Cloxacillin, Dicloxacillin, Methicillin, Oxacillin, Penicillin G, Penicillin V, Piperacillin Tazobactam, Ticarcillin Clavulanic Acid, Nafcillin, Cephalosporin I Generation, Cefadroxil, Cefazolin, Cephalexin, Cephalothin, Cephapirin, Cephradine, Cefaclor, Cefamandol, Cefonicid, Cefotetan, Cefoxitin, Cefprozil, Ceftinetazole, Cefuroxime, Loracarbef, Cefdinir, Ceftibuten, Cefoperazone, Cefixime, Cefotaxime, Cefpodoxime proxetil, Ceftazidime, Ceftizoxime, Ceftriaxone, Cefepime, Azithromycin, Clarithromycin, Clindamycin, Dirithromycin, Erythromycin, Lincomycin, Troleandomycin, Cinoxacin, Ciprofloxacin, Enoxacin, Gatifloxacin, Grepafloxacin, Levofloxacin, Lomefloxacin, Moxifloxacin, Nalidixic acid, Norfloxacin, Ofloxacin, Sparfloxacin, Trovafloxacin, Oxolinic acid, Gemifloxacin, Pefloxacin, Imipenem-Cilastatin, Meropenem, Aztreonam, Amikacin, Gentamicin, Kanamycin, Neomycin, Netilmicin, Streptomycin, Tobramycin, Paromomycin, Teicoplanin, Vancomycin, Demeclocycline, Doxycycline, Methacycline, Minocycline, Oxytetracycline, Tetracycline, Chlortetracycline, Mafenide, Silver Sulfadiazine, Sulfacetamide, Sulfadiazine, Sulfamethoxazole, Sulfasalazine, Sulfisoxazole, Trimethoprim-Sulfamethoxazole, Sulfamethizole, Rifabutin, Rifampin, Rifapentine, Linezolid, Streptogramins, Quinopristin Dalfopristin, Bacitracin, Chloramphenicol, Fosfomycin, Isoniazid, Methenamine, Metronidazol, Mupirocin, Nitrofurantoin, Nitrofurazone, Novobiocin, Polymyxin, Spectinomycin, Trimethoprim, Colistin, Cycloserine, Capreomycin, Ethionamide, Pyrazinamide, Para-aminosalicyclic acid, Erythromycin ethylsuccinate, Miconazole, Ketoconazole, Clotrimazole, Econazole, Bifonazole, Butoconazole, Fenticonazole, Isoconazole, Oxiconazole, Sertaconazole, Sulconazole, Tioconazole, Fluconazole, Itraconazole, Isavuconazole, Ravuconazole, Posaconazole, Voriconazole, Terconazole, Terbinafine, Amorolfine, Naftifine, Butenafine, Anidulafungin, Caspofungin, Micafungin, Benzoic acid, Ciclopirox, Tolnaftate, Undecylenic acid, Flucytosine, or 5-fluorocytosine, Griseofulvin, Haloprogin and combinations thereof. In one example, the antibiotic may include, but is not limited to nalidixic acid, gentamicin, erythromycin, streptomycin and kanamycin.

The terms “decrease”, “reduced”, “reduction”, “decrease”, “removal” or “inhibit” are all used herein generally to mean a decrease by a statistically significant amount. However, for avoidance of doubt, “reduced”, “reduction” or “decrease”, “removal”, or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level (e.g., in the absence of a peptide as described herein).

In another aspect, there is provided the use of a peptide of the present disclosure in the manufacture of a medicament for treating bacterial infection, or removing bacteria, or neturalising endotoxins, or treating a fungal infection or infestations, or removing fungus. In one example, the use may further comprise providing the peptide of the present disclosure for administration into a subject in need thereof. In one example, wherein the medicament is to be administered into a subject in need thereof.

In one example, the subject or patient may be an animal, mammal, human, including, without limitation, animals classed as bovine, porcine, equine, canine, lupine, feline, murine, ovine, avian, piscine, caprine, corvine, acrine, or delphine. In one example, the patient may be a human.

In one example, the peptide as described herein may be provided as a composition or a pharmaceutical composition. The compositions as described herein may be administered in a number of ways depending upon whether local or systemic treatment is desired. Administration may be topical, pulmonary (e.g., by inhalation or insufflation of powders or aerosols, including by nebulizer; intratracheal, intranasal, epidermal and transdermal) or systemic such as oral, and/or parenteral. Parenteral administration includes intravenous, intraarterial, subcutaneous, intraperitoneal or intramuscular injection or infusion; or intracranial, e.g., intrathecal or intraventricular, administration. In one example, the route of Administration may be selected from the group consisting of systemic administration, oral administration, intravenous administration and parenteral administration

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Compositions as described herein include, but are not limited to, solutions, pastes, ointment, creams, hydrogels, emulsions, liposome-containing formulations, and coatings. These compositions may be generated from a variety of components that include, but are not limited to, preformed liquids, self-emulsifying solids and self-emulsifying semisolids.

The formulations as described herein, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions as described herein may be formulated into any of many possible dosage forms including, but not limited to tablets, capsules, liquid syrups, soft gels, suppositories, and enemas. The compositions as described herein may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one example, the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

The compositions as described herein may additionally contain other adjunct components conventionally found in pharmaceutical compositions. Thus, for example, the compositions may contain additional, compatible, pharmaceutically-active materials such as, for example, antipruritic, astringents, local anaesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colourings, flavourings and/or aromatic substances and the like which do not deleteriously interact with the peptide(s) of the formulation.

The term “pharmaceutically effective amount” as used herein includes within its meaning a sufficient but non-toxic amount of the compound as described herein to provide the desired effect, that is, causing a Log reduction in the number of microorganisms of at least 1.0, which means that less than 1 microorganism in 10 remains. The modified peptides of the present disclosure may provide Log reductions in the number of microorganisms of at least about 2.0, or at least about 3.0, or at least about 4.0, or at least about 5.0, or at least about 6.0, or at least about 7.0. The exact amount required will vary from subject to subject depending on factors such as the species being treated, the age and general condition of the subject, the severity of the condition being treated, the particular agent being administered, the mode of administration, and so forth. Thus, it is not possible to specify an exact “effective amount”. However, for any given case, an appropriate “effective amount” may be determined by one of ordinary skill in the art using only routine experimentation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of the composition, and can generally be estimated based on EC50s found to be effective in in vitro and in vivo animal models or based on the examples described herein. In general, dosage is from 0.01 μg to 100 g/kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the composition is administered in maintenance doses, ranging from 0.01 μg to 100 g/kg of body weight, once or more daily, to once every 2 years.

In one example, the composition may be administered in an amount of between any one of about 0.01 μg, 0.05 μg, 0.1 μg, 0.5 μg, 1 μg, 5 μg, 10 μg, 20 μg, 30 μg, 40 μg, 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, 210 μg, 220 μg, 230 μg, 240 μg, 250 μg, 260 μg, 270 μg, 280 μg, 290 μg, 500 μg, 1 mg, 1.5 mg, 2 mg, 2.5 mg, 3 mg, 3.5 mg, 4 mg, 5 mg, 10 mg, 15 mg, 20 mg, 25 mg, 30 mg, 35 mg, 40 mg, 45 mg, 50 mg, 75 mg, 100 mg, 125 mg, 150 mg, 175 mg, 200 mg, 225 mg, 250 mg to any one of about 0.01 μg, 0.05 μg, 0.1 μg, 0.5 μg, 1 μg, 5 μg, 10 μg, 20 μg, 30 μg, 40 μg, 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, 210 μg, 220 μg, 230 μg, 240 μg, 250 μg, 260 μg, 270 μg, 280 μg, 290 μg, 500 μg, 1 mg, 1.5 mg, 2 mg, 2.5 mg, 3 mg, 3.5 mg, 4 mg, 5 mg, 10 mg, 15 mg, 20 mg, 25 mg, 30 mg, 35 mg, 40 mg, 45 mg, 50 mg, 75 mg, 100 mg, 125 mg, 150 mg, 175 mg, 200 mg, 225 mg, 250 mg, 300 mg/kg of body weight of the patient.

In one example, the concentration of the administered composition is about 1 to about 100 mg/Kg of body weight of the patient, about 5 to about 100 mg/Kg of body weight of the patient, about 10 to about 100-mg/Kg of body weight of the patient, about 20 to about 100 mg/Kg of body weight of the patient, about 30 to about 100 mg/Kg of body weight of the patient, about 1 to about 50 mg/Kg of body weight of the patient, about 5 to about 50 mg/Kg of body weight of the patient and about 10 to about 50 mg/Kg of body weight of the patient.

As used herein, the term “about”, in the context of amounts or concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

As used herein the term “consisting essentially of” refers to those elements required for a given example. The term permits the presence of additional elements that do not materially affect the basic and novel or functional characteristic(s) of that example of the invention.

The term “consisting of” refers to compositions, methods, and respective components thereof as described herein, which are exclusive of any element not recited in that given example.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

### Example 1

### Susceptibility Testings—Basic Screening

### Materials and Methods:

V2-dimer and all lipopeptides (FIG. 1A) used in this study were purchased from Mimotopes. All peptides were prepared by dissolving in sterile water to make 1000 μg/mL stock solutions. Minimum Inhibitory Concentration (MIC) determination was carried out in Mueller Hinton Broth (MHB) using the broth macro-dilution method as described by the Clinical and Laboratory Standards Institute (CLSI). Then, cation-adjusted MHB (CA-MHB) was used to prepare serial two fold dilutions of the compounds in test tubes. The concentration of the inoculums suspension was also adjusted to approximately 5×10⁵ Colony Forming Units (CFU)/mL using MHB. Bacteria were incubated with the compounds for 24 hours at 35° C. prior to reading. In this study, Gram-positive strains used were Staphylococcus aureus DM4001 and Methicillin-resistant Staphylococcus aureus (MRSA) DM9808. Gram-negative strains used were Klebsiella pneumoniae DM4299, Pseudomonas aeruginosa ATCC23155 and Escherichia coli ATCC27922.

TABLE 1A
Antibacterial activity: MIC values of V2D,
lipid modified V2D (C2-C14)

| | MIC (uM) | | | | |
|---|---|---|---|---|---|
| | | | K. | P. | |
| | S. aureus | MRSA | pneumoniae | aeruginosa | E. coli |
| Peptide | DM4001 | DM9808 | DM4299 | ATCC23155 | ATCC27922 |
| V2D | 5.46 | 2.73 | 5.46 | 5.46 | 2.73 |
| C2-V2D | 5.26 | 2.63 | 5.26 | 5.26 | 2.63 |
| C4-V2D | 2.57 | 2.57 | 5.14 | 10.28 | 2.57 |
| C6-V2D | 2.51 | 1.26 | 5.03 | 5.03 | 2.51 |
| C8-V2D | 2.46 | 1.23 | 2.46 | 2.46 | 4.92 |
| C10- | 2.40 | 1.20 | 2.40 | 2.40 | 4.80 |
| V2D | | | | | |
| C12- | 4.71 | 4.71 | 4.71 | 4.71 | 4.71 |
| V2D | | | | | |
| C14- | 2.30 | 2.30 | 9.22 | 2.30 | 1.15 |
| V2D | | | | | |
| C16- | 36.13 | 18.07 | >36.13 | >36.13 | 9.03 |
| V2D | | | | | |

TABLE 1B
Antibacterial activity: MIC values of G2D,
lipid modified G2D (C2-C14)

| | MIC (uM) | | | |
|---|---|---|---|---|
| | S. | | K. | P. |
| | aureus | MRSA | pneumoniae | aeruginosa |
| Peptide | DM4001 | DM9808 | DM4299 | ATCC23155 |
| G2D | 5.66 | 5.89 | 2.94 | 2.94 |
| C2-G2D | 5.52 | 5.66 | 5.66 | 2.83 |
| C4-G2D | 5.39 | 5.52 | 5.52 | 2.76 |
| C6-G2D | 5.26 | 2.70 | 2.70 | 5.39 |
| C8-G2D | 2.57 | 2.63 | 2.63 | 2.63 |
| C10-G2D | 2.51 | 2.57 | 2.57 | 2.57 |
| C12-G2D | >40.21 | 40.21 | 40.21 | >40.21 |
| C14-G2D | ND ^{[a]} | ND | ND | ND |
| C16-G2D | 5.66 | 5.89 | 2.94 | 2.94 |

^{[a]} Not determined

Compared with V2D, C8-V2D and C10-V2D show improved antimicrobial activities against Gram-positive and Gram-negative bacteria (Table 1A). Compared with G2D, antimicrobial properties of C8-G2D and V10-G2D show improved antimicrobial properties against Gram-positive bacteria (Table 1B).

### Example 2

### Susceptibility Testings—Further Screening for V2D, C8-V2D AND C10-V2D

### Materials and Methods:

Bacteria used in this study are listed in Table 2A and 2B below. Methods used are as described in Example 1 above.

### Results:

TABLE 2A
Antibacterial activity of V2D, C8-V2D and C10-V2D against a panel
of Methicillin-sensitive Staphylococcus aureus (MSSA)
and Methicillin-resistant Staphylococcus aureus strains.

| Organisms | V2D | C8-V2D | C10-V2D |
|---|---|---|---|
| MRSA DR 42412 | 2.73 | 1.23 | 1.20 |
| MRSA DM 21455 | 2.73 | 1.23 | 1.20 |
| MRSA 9808R | 2.73 | 1.23 | 1.20 |
| MRSA DB 57964 | 2.73 | 2.46 | 1.20 |
| MRSA DB 68004 | 2.73 | 2.46 | 1.20 |
| MRSA DB 21595 | 1.36 | 1.23 | 2.40 |
| MRSA DB 6506 | 2.73 | 1.23 | 1.20 |
| MRSA 43300 | 1.36 | 2.46 | 1.20 |
| MRSA 700699 | 2.73 | 1.23 | 1.20 |
| MSSA DM 0004583R | 2.73 | 2.46 | 2.40 |
| MSSA 0004001R | 5.46 | 2.46 | 2.40 |
| MSSA DM 4299 | 5.46 | 1.23 | 4.81 |
| MSSA DM 4400R | 2.73 | 2.46 | 2.40 |
| MSSA 29737 | 2.73 | 1.23 | 1.20 |
| MSSA 29213 | 2.73 | 2.46 | 2.40 |
| MSSA 6538 | 2.73 | 1.23 | 1.20 |

TABLE 2B
Antibacterial activity of V2D, C8-V2D and C10-V2D
against a panel of MSSA and MRSA strains.

| Organisms | V2D | C8-V2D | C10-V2D |
|---|---|---|---|
| PA DR 18531 | 2.73 | 1.23 | 1.20 |
| PA DM 4150R | 2.73 | 2.46 | 1.20 |
| PA DR 23257 | 2.73 | 2.46 | 2.40 |
| PA DR 23376 | 5.46 | 2.46 | 2.40 |
| PA DR 14476 | 5.46 | 2.46 | 2.40 |
| PA DR 23155 | 5.46 | 2.46 | 2.40 |
| PA DR 5790 | 10.91 | 2.46 | 2.40 |
| PA DR 4877 | 5.46 | 2.46 | 4.81 |
| PA ATCC 9027 | 5.46 | 2.46 | 2.40 |
| PA ATCC 27853 | 5.46 | 2.46 | 2.40 |

S. aureus (MSSA and MRSA):

Total strains tested: 16
i) C8V2D-Improved 2-fold for 8 strains. Improved 4-fold for 1 strain.
   % Improvement=56.3%
ii) C10V2D-improved 2-fold for 10 strains.
   % Improvement=62.5%

P. aeruginosa:

Total Strains tested: 10
i) C8V2D-Improved 2-fold for 7 strains. Improved 4-fold for 1 strain
   % Improvement=80%
ii) C10V2D-improved 2-fold for 7 strains. Improved 4-fold for 1 strain
   % Improvement=80%

### Example 3

### Time-Kill Kinetics

### Materials and Methods

Bacterial used in time-kill studies were isolated from the 18-20 hours Tryptic Soy Agar (TSA) plate. The inoculum was then suspended and adjusted in 0.31 mM phosphate buffer to obtain the bacterial suspension with 10⁵ to 10⁶ CFU/mL. Then, the inoculum was treated with various concentration of C8V2D. The mixtures were incubated at 35° C. Culture aliquots were removed at 0, 10 min, 30 min, 1 h, 2 h, 5 h and 24 h for viable plate counts. The aliquots were 10-fold serially diluted using Dey-Engley (D/E) Neutralization Broth, and then a 20 μL of each dilution was plated out on TSA plates using the surface-spread plate method. The plates were then incubated at 35° C. for 48 to 72 h. Cell viability was assessed by enumerating the colonies grown on the plates. Bactericidal activity was defined as 3-log reduction of viable count in culture treated with antimicrobial compared with the untreated control at the start of each assay. To assess the possibility of carry-over of the antimicrobial agents onto the plates tested strains as control in the serial dilutions in the presence and absence of 5c and 6 were examined. Bacterium used in this study was P. aeruginosa ATCC23155.

As seen in FIG. 1, 3-log reduction in 20-30 min at 2× and 4×MIC was observed in the in vitro time-kill kinetic assay of the present Example. Thus, demonstrating that C8V2D could kill Pseudomonas aeruginosa rapidly.

### Example 4

### In Vitro and In Vivo Toxicity

### 4A. Hemolytic Assay

### Materials and Methods

Fresh red blood cells (RBCs) of New Zealand white rabbits were used for this experiment. The RBCs obtained were centrifuged at 3000 rpm for 10 min. Supernatant was removed and washed twice with sterile PBS Buffer (20 mM, 100 mM NaCl, pH 7). The RBCs were further diluted to 8% stock solution in PBS. Peptides were dissolved in PBS with desired stock concentrations. When compounds were mixed with RBCs, desired concentration of 4% RBCs was obtained. For C14V2D and C16V2D, they were dissolved in Dimethylformamide ((CH₃)₂NCH; DMF) with desired stock concentrations. When compounds were mixed with RBCs, desired concentration of 4% RBCs and 0.5% DMF was obtained. DMF concentration of <1% was used as it had negligible hemolytic activity on RBCs. The mixtures were added to 1.5 mL centrifuge tubes and incubated at 37° C. for 1 h. After incubation, the blood was centrifuged at 3000 rpm for 3 min at 4° C. 100 μL of the supernatant was transferred into transparent 96-well plate. The absorbance was measured at 576 nm using a microplate reader (TECAN Infinite 200M Pro). 2% Triton X-100 was used as positive control. PBS was used as negative controls. The amount of hemoglobin released was calculated using the following equation:

$$\% Hemolysis = \frac{Mixture {abs}_{576 nm} - Negative control {abs}_{576 nm}}{Positive control {abs}_{576 nm} - Negative control {abs}_{576 nm}} \times 100$$

Results:

TABLE 3A
Hemolytic activity of peptide
and lipid-modified peptide dimers.

| Compound | HC10 (ug/mL) | Selectivity^{d} |
|---|---|---|
| V2D | >2000 | >800 to >1600 |
| C2V2D | >2000 | >800 to >1600 |
| C4V2D | >2000 | >400 to >1600 |
| C6V2D | >2000 | >800 to >3200 |
| C8V2D | >2000 | >800 to >3200 |
| C10V2D | >2000^{a} | > 800 |
| C12V2D | 542.8 ± 53.0 | 43.4 |
| C14V2D | ^{b}251.82 ± 5.2 | 10-81 |
| C16V2D | ^{c}114.0 ± 37.9 | 1-2 |

^{a}% Hemolysis = 3.3% at 20 mg/mL.
^{b}Value in PBS. HC10 in DMF is 173.3 ± 35.2 mg/mL.
^{c}Value in DMF. C16V2D could not dissolve in PBS.
^{d}Selectivity = HC10/MIC

HC10 is concentration of the peptide to induce 10% of haemoglobin released from the red blood cells. Table 3A shows that V2D and C2-C10 V2D were non-hemolytic up to 2000 μg/mL. Thus, V2D and C2-C10 V2D are very specific toward bacterial membrane. C12V2D displays more potent haemolytic activity, and C14 and C16V2D caused significant hemolysis at lower concentration. In general, haemolytic activity increase with lipid length coupled to the peptide dimers, and therefore the selectivity reduced with increase of lipid length.

### 4B. In Vitro Toxicity Tests.

### Lactate Dehydrogenase Assay

The cytotoxicity of individual compounds screened was determined by the lactate dehydrogenase (LDH) assay. In brief, human corneal fibroblast cells were plated at a density of 10,000 cells per well in a 96-well opaque white plate (SPL Life Sciences Inc). Test compounds of various concentrations and controls were added to appropriate wells such that the final volume is 100 μl in each well. After 4 hours of exposure to the test compounds, the plates were removed from 37° C. incubator and equilibrated to 22° C. for 30 minutes. One hundred microliters of Cyto-TOX One reagent (Promega Inc. USA) was added to each well and the LDH assay was performed according to manufacturer's instructions. Fluorescence was assessed at an excitation wavelength of 560 nm and an emission wavelength of 590 nm using a micro-plate reader (Tecan Infinite 200 Pro, Switzerland). The cells exposed to 1% Triton X-100 were used as a positive control and treated as maximum releasing of LDH. The percent specific cytotoxicity of each compound was determined using the formula below (I=intensity):

% Cytotoxicity=[(Iₑₓₚₑᵣᵢₘₑₙₜₛ)−(I_{negative controls})]/[I_{positive controls})−(I_{negative controls})]×100

### 4C. Cell Viability (ATP) Assay

Cells were plated at a density of 10,000 cells per well in a 96-well opaque white plate (SPL Life Sciences Inc. Korea). Test compounds of various concentrations and controls were added to appropriate wells such that the final volume is 100 μl in each well. After 4 hours of exposure to the test compounds, the plates were removed from 37° C. incubator and equilibrated to 22° C. for 30 minutes. One hundred microliters of CellTiter-Glo reagent (Promega Inc. USA) was added to each well and the ATP assay was performed according to manufacturer's instructions. Luminescence was assessed using a micro-plate reader (Tecan Infinite 200 Pro, Switzerland). The cells exposed to 1% Triton X-100 were used as a positive control. The percent cell viable of each compound was determined using the formula below (L=Luminescence detected):

% Viability=[(L_{negative controls})−(Lₑₓₚₑᵣᵢₘₑₙₜₛ)]/[(L_{negative controls})−(L_{positive controls})]×100

### Results:

TABLE 3B
In vitro toxicity of C8V2D compared to V2D.

| | TC50 (uM)^{[a]} | |
|---|---|---|
| Compounds | LDH | ATP |
| V2D | >1000 | >1000 |
| C8V2D | >3000 | >2000 |

^{[a]}Toxicity concentration (TC50) to induce 50% of cytotoxicity and derease 50% of cell viable.

### 4D. In Vivo Topical and Acute Toxicity Tests.

### Materials and Methods:

Wild type C57BL6 (6-8 weeks old) (20-30 gram weigh) mice purchased from National University of Singapore were used for this study. All animals were utilized after 1 week acclimatization and put in air conditioned rooms with controlled temperature (23±2° C.), 12 hours light-dark cycle and humidity (55-60%). All animals were conducted in compliance with the Association for Research in Vision and Ophthalmology (ARVO) statement for the Use of Animals in Ophthalmic, and Vision Research, the guide for the Care and Use of laboratory animals (National Research Council) and under the supervision of Singhealth Experimental Medical Centre (SEMC). To investigate the corneal toxicity, three normal and healthy wild type mice had been chosen randomly and treated with 1 mg/ml and 3 mg/ml of C8V2D in 10 mM PBS. C8V2D was applied topically 5 times/day for 4 days. Cornea clarity had been checked by slit lamp microscopy for everyday follow-up to day 4 after application. To study the acute systemic toxicity, C8V2D was administrated via intravenous and intraperitoneal routes. Two mice have been chosen for each route and moitored carefully through 24 hours to observe and record any mortality, morbidity or toxicity signs. Gross necropsy would be done on any mortality or moribund animal.

### Results

TABLE 3C
Safety concentration of C8V2D in in
vivo topical and acute toxicity tests.

| Route | Tolerate concentration | |
|---|---|---|
| Topical | ≧3 | mg/kg |
| Intravenous (i.v.) | 6.25 | mg/kg |
| Intraperitoneal (i.p.) | 100 | mg/kg |

Both in vitro and in vivo toxicity profile shows that C8V2D is safe. C8V2D is a novel broad spectrum antimicrobial, which is non-toxic based on in vitro and in vivo studies.

### Example 5

### Lipopolysaccharides (LPS) Neutralization Study

### To Study the Possible Effect of Peptide and Lipid-Modified Peptide of the Present Disclosure to the Lps of Gram-Negative Bacteria

### Materials and Methods

The lipopolysaccharide (LPS) neutralization was assessed using pierce Limulus Amoebocyte Lysate (LAL) chromogenic endotoxin quantitation kit (Thermo Scientific) with minor modifications to the manufacturer's protocol. LAL contains enzymes that are activated in a series of reactions in the presence of LPS. The last enzyme activated in the cascade splits the chromophore into para-nitro aniline (PNA), from the chromogenic substrate and produces a yellow colour. The amount of PNA released will be measured photometrically at 405 nm which is proportional to the amount of LPS in the system. Briefly, the microplate was first equilibrated in a heating block for 10 minutes at 37° C. Then, the desired concentrations of lipid-modified peptides were pipetted into the microplate well and incubated for 5 minutes at 37° C. The mixtures were then incubated with LAL at 37° C. for 10 mins followed by the incubation with 2 mM substrate solution at 37° C. After 10 mins, 25% of acetic acid was added into each well to stop the reaction and the absorbance was measured at 405-410 nm. Effective concentration to neutralize 50% of LPS (NC50) was then determined. In this study, LPS from Escherichia coli 0111:B4 (Sigma Aldrich) and LPS from P. aeruginosa 10 (Sigma Aldrich) were used.

### Results

TABLE 4A
Value of effective concentration of V2D and lipid modified
V2D to neutralize 50% of LPS of E. coli

| Compound | NC50 |
|---|---|
| V2D | 1452 |
| C2V2D | 1295 |
| C4V2D | 2000 |
| C6V2D | 187 |
| C8V2D | 73 |
| C10V2D | 38 |
| C12V2D | 29 |
| C14V2D | 32 |
| C16V2D | 48 |

TABLE 4B
Value of effective concentration of V2D and lipid modified V2D
to neutralize 50% of LPS (NC50) of P. aeruginosa

| Compound | NC50 |
|---|---|
| V2D | 693.8 |
| C2V2D | >2000 |
| C4V2D | >2000 |
| C6V2D | 447.3 |
| C8V2D | 266.9 |
| C10V2D | 216.3 |
| C12V2D | 78.4 |
| C14V2D | 44.1 |
| C16V2D | 45.1 |

### Result:

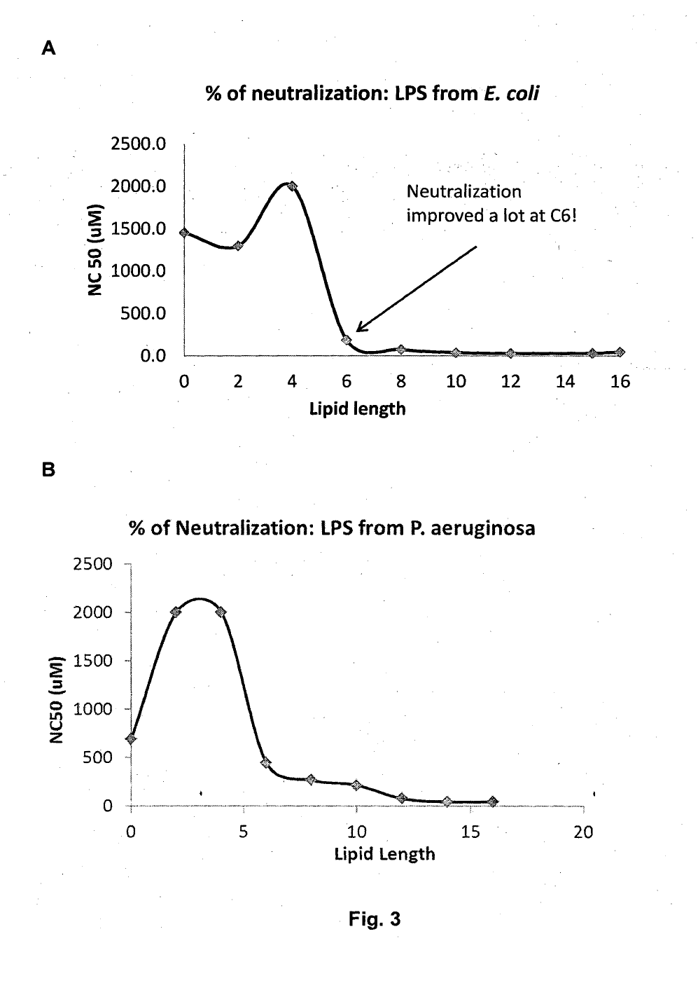

Lipid-modified peptides with longer lipid length (n≧6) could neutralize the LPS efficiently. The critical chain length to neutralize the LPS significantly was n=6. For LPS of E. coli, the improvements of LPS neutralization compared to V2D were: C6V2D=7.8-fold; C8V2D=20-fold and C10V2D=38 fold (See FIG. 3A and Table 4A).

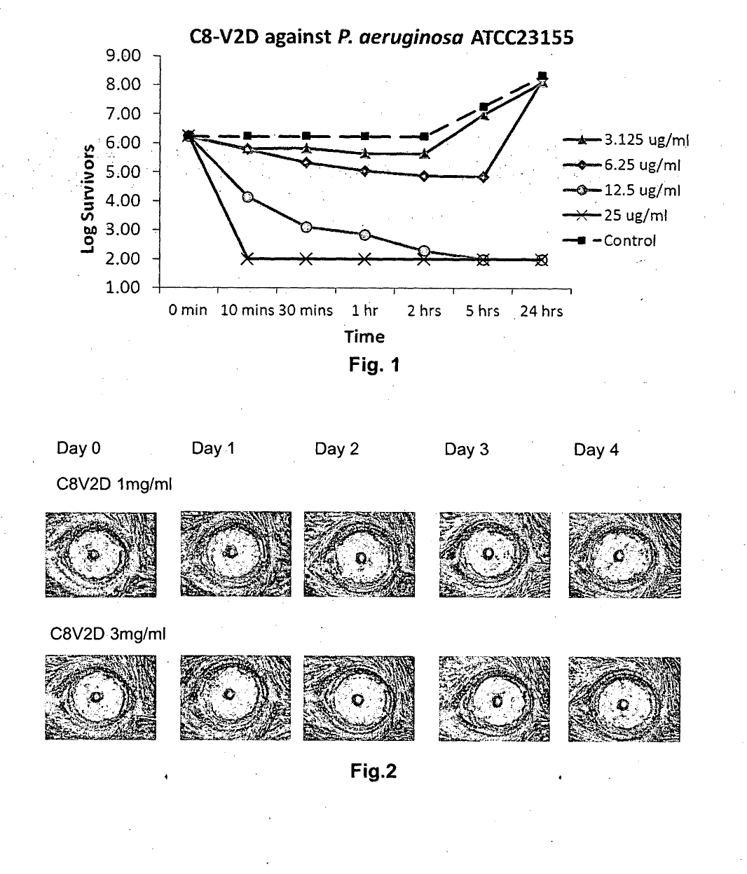

Similar pattern was obtained for neutralization of LPS of P. aeruginosa (FIG. 2B and Table 4B). However, the concentrations required to neutralize 50% of P. aeruginosa LPS were higher than E. coli LPS. The improvements of LPS neutralization compared to V2D were: C6V2D=1.6-fold; C8V2D=2.6-fold and C10V2D=3.2-fold (See FIG. 3A and Table 4A).

### Example 6

### LPS-Bodipy Displacement Assay to Study the Binding Ability of Lipid-Modified Peptides with Lipopolysaccharides

Bodipy TR cadaverine (BC) is a fluorescent probe used in LPS binding assay. The binding of BC to the lipid A moiety of LPS forms a complex via ionic bridges. Outer-membrane permeabilisers like Polymyxin B will displace BC from lipid A of LPS leading to de-quenching effect which will cause the fluorescence intensity to increase. Bodipy TR cadaverine was dissolved in DMF and diluted with TRIS buffer (50 mM, pH 7.4). Equal volumes of 100 μg/mL LPS and 10 μM Bodipy TR Cadaverine were mixed together and aliquots of the mixture were added to the desired concentrations of lipid-modified peptides in a SPL 96 Black well plate (SPL Life Sciences). 40 μM Polymyxin B was used as a positive control in the experiment. Fluorescence readings were measured by TECAN infinite 200 microplate reader at excitation wavelength of 580 nm and emission wavelength of 620 nm.

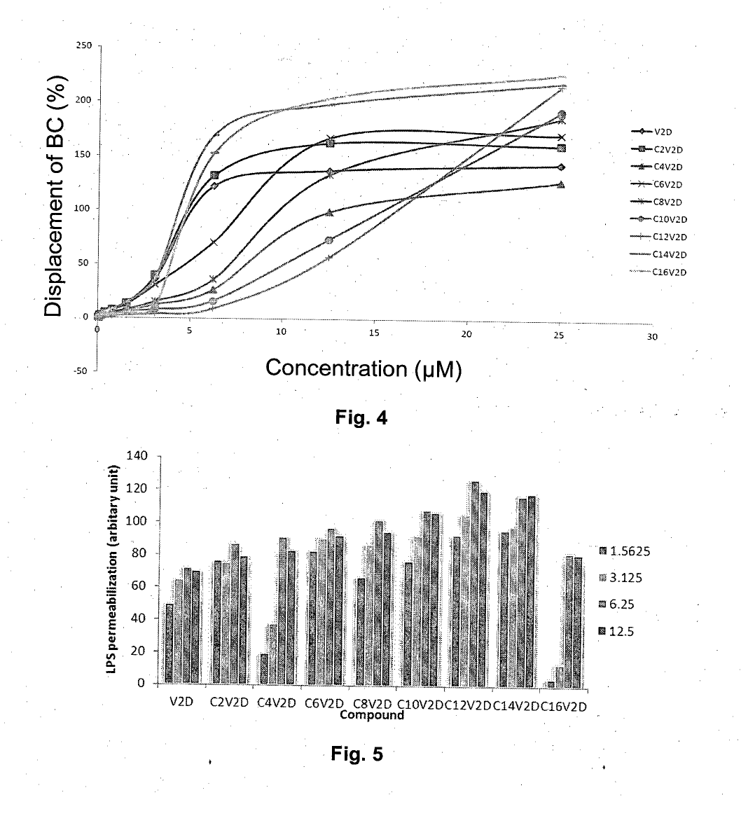

FIG. 4 shows the displacement of BC from Bodipy by peptides and lipid-modified peptides of the present disclosure. In general, sigmoidal curves were obtained for all the peptides LPS binding is stronger for C8-C16V2D (graph have not plateau yet). Data shows that lipid peptides can bind to LPS and displace Bodipy from Lipid A

### Example 7

### NPN Outer Membrane Permeabilization Assay to Study the Effect of Peptide and Lipopeptide on Permeabilization of Outer Membrane or LPS

### Materials and Methods

Due to the presence of lipopolysaccharide (LPS) molecules constituting the outer leaflet of the outer membrane, the outer membrane is able to prevent entry of external hydrophobic molecules such as 1-N-phenylnaphthylamine (NPN). To investigate whether the antimicrobial lipid-modified peptides (lipopeptides) could permeabilise the outer-membrane of bacteria, NPN which shows high fluorescence in phospholipid environment but fluoresces poorly in aqueous environment was used. Clinical isolate E. coli ATCC 8739 was collected at an early exponential growth phase and suspended in 5 mM HEPES buffer solution (2 mM EDTA at pH 7) until an optical density of 0.35 at 670 nm [OD630] was obtained. 40 μM NPN stock solution (C₁₀H₇NHC₆H₅) was prepared by dissolving NPN in acetone and diluted with 5 mM HEPES buffer. Bacterial suspensions were then added to 40 μM NPN solution in a 96 Black well plate (SPL Life Sciences). The desired concentrations of lipopeptides were added to the well plates and mixed thoroughly. The final concentration of NPN in the well plate was 20 μM. The addition of 5 mM HEPES was used as a negative control in the experiment. Fluorescence readings were measured by TECAN infinite 200 microplate reader at excitation wavelength of 355 nm and emission wavelength of 405 nm.

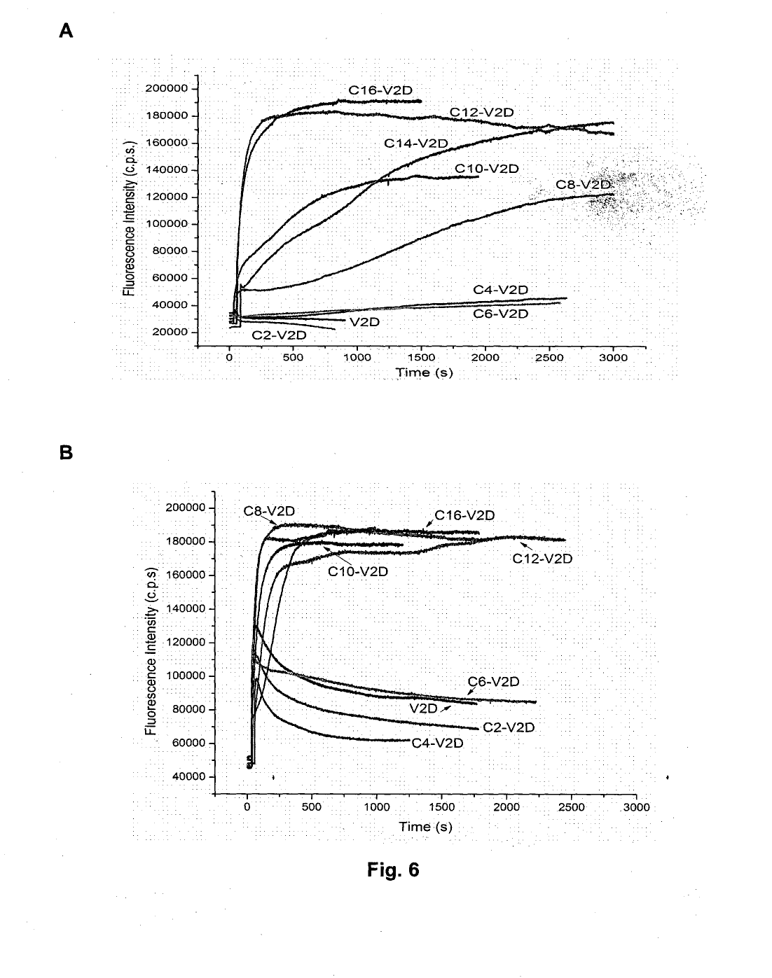

Results: As observed in FIG. 5, NPN assay shows that the lipid-modified peptides of the present disclosure could permeabilize lipopolysaccharides. C8V2D-C14V2D have better permeabilization effects than V2D.

### Example 8

### Synergistic Studies of Lipopeptides with Commercially Available Antibiotics

### Aim: To Study the Significant of Lps Neutralization of Lipopeptides as a Synergistic Agent

### Materials and Methods

The synergistic interactions of V2D, C8V2D and C10V2D with other commercially available antibiotics were determined using checkerboard microdilution methods with MHB. Briefly, MICs of each antibiotics used in synergy studies were determined by broth microdilution method. The range of concentrations was prepared according to the MIC of each compound and each antibiotic which were determined earlier. Concentration range tested ranged from 1×MIC to 0.03×MIC (or further diluted until a minimum Total Fractional Inhibitory Concentration, FIC was obtained). The two-fold dilutions of compounds and antibiotics were prepared and mixed in sterilized test tubes. Then, suspensions containing bacteria were added forming a final inoculums of approximately 5×10⁵ CFU/mL. The test tubes were then incubated for 24 hours at 35° C. An aliquot (200 μL) from each test tube was added to a sterile 96-well flat bottom plate (SPL Life Sciences). MIC90 was determined using TECAN Infinite 200 microplate reader by measuring the absorbance at optical density at 600 nm. The antibiotics used were nalidixic acid, gentamicin, erythromycin, streptomycin and kanamycin. Total FIC was calculated using the formula according to the formula Σ FIC=FIC_{A}+FIC_{B}, where FIC_{A} or FIC_{B}=MIC agent A or B in combination/MIC of agent A or B alone. Σ FIC values were interpreted as follows: Σ FIC of values ≦0.5 denoted synergy, Σ FIC values of 0.5-0.75 denoted partial synergism and 0.75 to 4 denoted indifference and Σ FIC values of ≧4 denoted antagonism.

### Results:

TABLE 5
Fractional inhibition concentration index of V2D, C6V2D and C8V2D with 5 different
antibiotics against P. aeruginosa and E. coli

| | FIC | | | | | |
|---|---|---|---|---|---|---|
| | P. aeruginosa ATCC23155 | | | E. coli ATCC25922 | | |
| Compounds | V2D | C6V2D | C8V2D | V2D | C6V2D | C8V2D |
| Nalidixic Acid | 0.375 | 0.375 | 0.5 | 0.75 | 1 | 0.5 |
| Gentamicin | 0.3125 | 0.25 | 0.375 | 0.375 | 0.375 | 0.3125 |
| Erythromycin | 0.5 | 0.375 | 0.5625 | 1 | 0.375 | 0.5625 |
| Streptomycin | 0.3125 | 0.375 | 0.5625 | 0.5 | 0.375 | 0.3125 |
| Kanamycin | 0.375 | 0.3125 | 0.5625 | 0.625 | 0.5 | 0.3125 |

P. aeruginosa:

C6V2D has better synergism effect (3/5 antibiotics tested). C8V2D has weaker effect.

E. coli:

C8V2D has much better synergisim effect than V2D (5/5 antibiotics tested). C6V2D also shows good synergistic interaction too.

The lipid-modified peptides of the present disclosure was also believed to act at sub-microgram and sub MIC (minimum inhibitory concentration) values to increase activity of existing antibiotics even on resistant forms of Pseudomonas. The synergistic effect is illustrated in the following Tables.

### Example 9

### Inner Membrane Targeting Properties of Lipopeptides

### Aim: To Study the Inner Membrane Interactions of Lipopeptides with Gram-Positive and Gram-Negative Bacteria

### 9A. DisC3-5 Cytoplasmic Membrane Depolarization Assay

### Materials and Methods

2.5 Cytoplasmic Membrane Depolarization Assay

DisC3-5 (3,3′-dipropylthiadicarbocyanide iodide, Invitrogen) is a membrane potential sensitive dye that is partitioned between the cells and medium according to the cytoplasmic membrane potential of the bacterial cell. Partitioning of DisC3-5 onto polarized cytoplasmic membrane would self-quench the fluorescence intensity. The addition of a membrane active antimicrobial that dissipates the membrane potential will cause the dye to be released into the surrounding medium and the increase in fluorescence will be observed. The effect of lipopeptides on the membrane potential of isolate. S. aureus (DM4001) and E. coli ATCC8739 was investigated. Briefly, clinical isolate S. aureus (DM4001) or E. coli ATCC8739 was collected at an early exponential growth phase and suspended with 5 mM HEPES Buffer (0.1 M KCl and 0.2 mM EDTA at pH 7) until an optical density of 0.09 at 620 nm [OD620] was obtained. For lipopeptides, the cell suspension was incubated with 0.4 μM of DiSC3-5 at 37° C. for 30 minutes until DiSC3-5 uptake was maximal. Using a Photon Technology International Model 814 fluorescence spectrophotometer, the fluorescence reading was monitored for 500 seconds until a stable baseline was obtained at an excitation wavelength of 660 nm and an emission wavelength of 675 nm. The desired concentrations of the lipopeptides were added into a stirred cuvette and the fluorescence signal was monitored until the readings were stabilised.

### Results

FIG. 6 shows the effects of lipid-modified peptides of the present disclosure on the fluorescence intensity of DiSC3-5 in the presence of (A) clinical isolate of S. aureus DM4001 and (B) E. coli ATCC 8739. As demonstrated by FIG. 6, the longer the lipid tail length of the lipid-modified peptide is, the more DisC3-5 depolarisation observed. In fact, C8V2D to C16V2D were able to cause effective depolarisation of the bacterial cell membrane.

### 9B. Membrane Damaging Effect of Lipopeptide Analogues

### Materials and Methods:

The extent of bacterial membrane damage was assessed using LIVE/DEAD BacLight Bacterial Viability Kit (Molecular Probes, Invitrogen) which utilises both SYTO 9 green-fluorescent nucleic acid stain and the red-fluorescent nucleic acid stain, propidium iodide in their different ability to penetrate healthy bacterial cells. SYTO9 dye could penetrate bacteria with intact and damaged membrane but propidium iodide could only penetrate bacteria with damaged bacteria thereby causing a reduction in the SYTO 9 stain's fluorescence when both dyes are present. Thus, bacteria with intact cell membranes stain fluorescent green whereas bacteria with damaged membranes stain fluorescent red. Briefly, clinical isolate S. aureus (DM4001) was collected at an early exponential growth phase and washed twice with 0.9% saline solution (live culture). A portion of the culture was resuspended in 70% 2-propanol to prepare dead culture with fully permeabilised membrane. Both live and dead cultures were incubated at 37° C. for 1 h before re-suspending in 0.9% saline solution until an optical density of 0.30 at 670 nm [OD670] was obtained. The desired concentrations of lipopeptides were incubated with live culture suspension at 37° C. for 10 mins and 30 mins respectively. Then, an aliquot of the mixtures and standards were added to an equal volume of dye mixture (10 μM of SYTO 9 stain and 60 μM of propidium iodide) in a 96 Flat Black well plate (Corning Life Sciences) and mixed thoroughly. Using TECAN infinite 200 microplate reader, the wavelength for excitation was fixed at 485 nm to obtain the emission spectrum from 500 nm to 700 nm. The green to red ratio (G/R) was determined using the following formula:

$$\frac{G}{R} ratio = \frac{{\sum }_{{I}_{510 nm}}^{{I}_{540 nm}} {F}_{cell . em}}{{\sum }_{{I}_{620 nm}}^{{I}_{650 nm}} {F}_{cell . em}}$$

% of membrane damage was quantified using a G/R ratio standard curve generated using bacterial mixture of 0, 10%, 50%, 90% and 100% live culture suspension. The set of standard solutions were obtained by mixing different volumes of live culture suspension and dead culture suspension together.

### Results

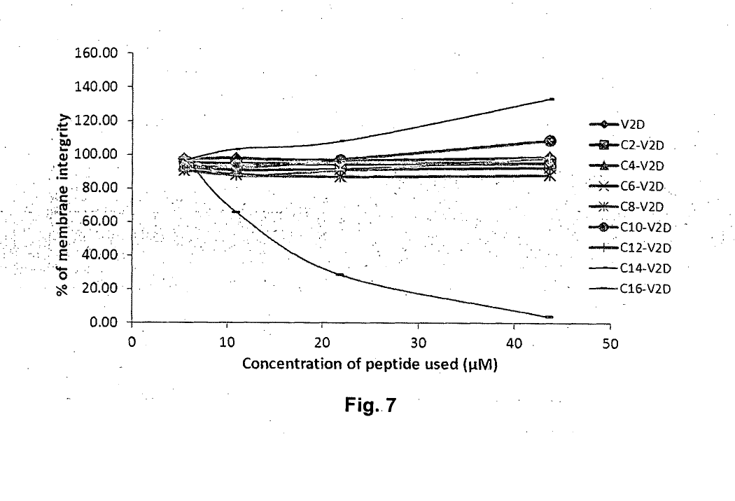

As shown in FIG. 7, no effect on membrane integrity was observed for V2D to C14V2D. However, reduced membrane integrity was observed for C16V2D at high concentrations (1×MIC and above). Thus, demonstrating that peptides modified with lipid group with 16 carbon length could cause disruption on the membrane integrity of bacteria. The results suggest that the permeabilisation of the bacterial cell membrane requires very long lipid tails (increased hydrophobicity). Therefore, whilst C8-C14V2D were shown to be able to depolarize bacterial membrane, disruption in membrane integrity requires longer lipid group.

### Example 10

### Membrane Selectivity Study Using Calcein Encapsulated Liposome

### Materials and Methods

Calcein at self-quenched concentration was hydrated with the liposome to encapsulate the calcein. The fluorescence intensity was observed to be low due to self-quenching. When the liposome is disrupted by the antimicrobials, the dye would be released and diluted by the solvent. This would decrease the extent of self-quenching and leads to the increase in fluorescence intensity. All phospholipids used in this assay were purchased from Avanti Polar. Lipids, Inc. (Alabaster) and used without further purification. Phospholipids used in this study were 1,2-di-(9Z-octadecenoyl)-sn-glycero-3-phosphoethanolamine (DOPE), 1,2-dioleoyl-sn-glycero-3-phospho-(1′-rac-glycerol) (sodium salt) (DOPG) and E. coli total lipid extract. The calcein loaded large unilamellar vesicles (LUVs) were prepared using film hydration method. Since the major components in the bacterial inner membrane are DOPE and DOPG, lipid molecules with a ratio of DOPE/DOPG=3/1 is a generally accepted model to mimic the general model of bacterial membrane. Similarly, the liposome composition of DOPC is a generally accepted model to mimic general model of RBC membrane to study the selectivity of the compound. The lipids DOPE/DOPG=3/1 or DOPC were dissolved in methanol/chloroform (1:2 by volume). The solvent was dried under nitrogen gas followed by freeze-drying for at least 1 hour. The dried lipid film was then hydrated with calcein solution (80 mM calcein, 50 mM HEPES buffer at pH 7) to a final lipid concentration of 30 mM. The hydrated lipid vesicles were subjected to freezing in liquid nitrogen and thawed in water bath for 7 cycles. Extrusion was carried out for 10 cycles using a polycarbonate membrane (Whatman, pore size 100 nm) in a mini-extruder (Avanti Polar Lipid Inc.) to prepare homogeneous LUVs of 100 nm. Using a gel filtration column using Sephadex G-50, calcein encapsulated vesicles were separated from free calcein. The concentration of eluted liposomes was determined by total phosphorus determination assay. An aliquot of the calcein encapsulated LUVs was transferred to a stirred cuvette. Then, desired concentrations of lipopeptides (dissolved in 50 mM HEPES) were added to obtain the desired lipid to xanthone analogue ratios of 1/2, 1/4 and 1/8 and desired lipid to lipopeptide ratios of 1/2, 1/4, 1/8, 1/16, 1/32, 1/64 and 1/128 respectively. Final concentration of liposome in the cuvette was 50 μM. 0.1% Triton X-100 was added as a positive control to determine the intensity at complete leakages. The fluorescence emission intensity was monitored using TECAN infinite 200 microplate reader at an excitation of 490 nm and am emission wavelength of 520 nm for 30 minutes. Percentages of leakage (% L) was calculated using the following formula: % L=[(Iₜ calₒ)/(I_{∞}−Iₒ)]×100%, where Iₒ and Iₜ are intensities before and after addition of xanthone analogues/lipopeptides respectively, and I_{∞} is intensity after addition of 0.1% Triton X-100.

### Results

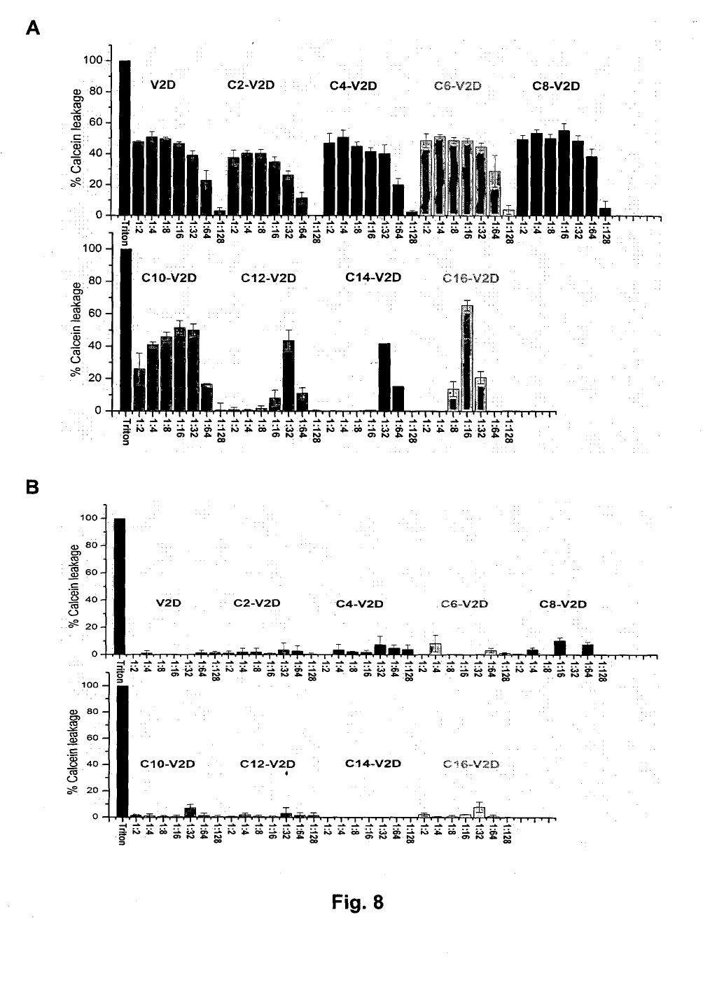

In the present disclosure, the interaction of the lipopeptides with lipid membrane was studied using artificial lipid membrane. In the present membrane selectivity study, lipid composition of DOPE/DOPG=3/1 was used to mimic bacterial membrane. The assay showed that lipopeptides of the present disclosure were able to induce ˜50% calcein leakage in bacterial inner membrane (FIG. 8A). To observe the selectivity of the lipid-modified peptides of the present disclosure, liposome which mimics red blood cell membrane was also constructed. FIG. 8B shows that the as compared to calcein leakage observed in liposome mimicking bacterial membrane (FIG. 8A), the lipid-modified peptides of the present disclosure did not cause significant red blood cell membrane disruption. Accordingly, the results of the present study suggest that DOPE is the main component for providing electrostatic interaction with the lipid-modified peptides, thus providing excellent selectivity of the lipid-modified peptides of the present disclosure towards bacterial membrane.

### Example 11

### Synergistic Studies on the Peptides of the Present Disclosure in Combination with an Antibiotic

### Determination of Minimum Inhibitory Concentration:

The minimum inhibitory concentration (MIC) was determined using the broth microdilution method. Bacterial cells (shown in Table 6) are grown in Mueller Hinton Broth (MHB) overnight. 100 μL of adjusted inoculum in MHB is added to 100 μL of each dilution of peptide or antibiotics dissolved in the broth, so as to yield a final cell density of 105 to 106 cfu/mL in each well. The plates were incubated at 35° C. for 24 h and the absorbance at 600 nm (OD600) was monitored every 30 minutes. A positive control well contained the broth and organisms (no peptides/antibiotics), and a negative control tube contains only the broth. The MIC of peptides for each clinical isolate or reference organism was recorded as the lowest concentration of peptide/antibiotic that inhibited visible growth of the test organism. To study the effect of Mg2+ on MIC of branched peptides, the concentration of MgCl2 was varied in MHB and the MIC was determined as before.

TABLE 6
Minimum inhibitory concentration
(MIC) of B2088 and B2088_99

| | MIC in μM of | |
|---|---|---|
| Strains^{a} | B2088 | B2088_99 |
| Pa DR 18531 | 2.73 | 2.9 |
| Pa DM 4150R | 2.73 | 5.8 |
| Pa DR 23257 | 2.73 | 5.8 |
| Pa DR 23376 | 5.46 | 5.8 |
| Pa DR 14476 | 5.46 | 5.8 |
| Pa DR 23155 | 2.7 | 2.9 |
| Pa DR 5790 | 10.91 | 5.8 |
| Pa DR 4877 | 2.7 | 2.9 |
| Pa ATCC 9027 | 2.7 | 2.9 |
| Pa 23155 | 2.7 | 2.9 |
| Pa ATCC 27853 | 2.7 | 2.9 |
| Kp ATCC 10031 | 1.4 | 1.5 |
| Kp 4299 | 2.7 | 2.9 |

^{a}Pa is Pseudomonas aeruginosa and Kp is Klebsiella pneumoniae

TABLE 7
Bactericidal properties of B2088 and B2088_99 as measured
by ED50 (Effective dose to kill 50% of bacterial cells)

| | MIC in μM of | |
|---|---|---|
| Strains | B2088 | B2088_99 |
| PA 9027 | 0.7 ± 0.02 | 0.33 ± 0.05 |
| PA 28753 | 1.1 ± 0.04 | 0.48 ± 0.03 |

### Determination of Fractional Inhibitory Concentration Index (FICI):

For the determination of FICI of peptides in combination with other antibiotics, the multidrug resistant strain, P. aeruginosa DR4877 was used. Prior to the testing, stock solutions of each drug (multivalent peptides and antibiotics) to at least 2×MIC are prepared in Mueller-Hinton broth (MHB). The checkerboard was assembled on a 96-well microtitre plate by overlaying serial two-fold dilutions of multivalent peptides perpendicularly (i.e. along the ordinate) to serial dilutions of antibiotics (i.e., along the abscissa). Each well consisted of 100 μL serially diluted peptide or antibiotics alone and in combinations in MHB and 100 μL inoculum (OD600=0.08). The microtitre plate was incubated at 35° C. for 24 h. Inhibition was determined both by visual examination and by OD600 measurements. The fractional inhibitory concentration indices will be calculated using the equation,

$${FIC}_{index} = \frac{{MIC}_{comb}^{peptide}}{{MIC}_{o}^{peptide}} + \frac{{MIC}_{comb}^{antibiotics}}{{MIC}_{0}^{antibiotics}} .$$

The FIC indices used to characterize antibiotic combinations as follows: FIC index<0.5 synergistic; additivity, 0.5<FIC index>1.0; indifference, 1<FIC index<4; FIC index>4, antagonism. The synergistic action of the peptides with polymyxin B as a standard was also compared.

Effect of LPS and Mg2+ on MIC: To examine the interaction of peptide and LPS, the concentration of the latter was added exogenously from (0.001-/100 μg/mL) in MHB at a peptide concentration of 1×MIC. The % inhibition was estimated and the amount of LPS required for 50% of antibacterial activity (IC50) was determined.

TABLE 8
Synergism between B2088 and B2088_99 with various classes of
antibiotics. The multidrug resistant strains P. aeruginosa
DR4877 was used for the experiments.

| | | FIC Index | |
|---|---|---|---|
| Antibiotics | Class | B2088 | B2088_99 |
| Carbenacillin | Penicillin | 0.5 | 0.63 |
| Chloramphenicol | | 0.38 | 0.38 |
| Erythromycin | Macrolides | 0.5 | 0.63 |
| Nalidixic acid | Quinalolnes | 0.56 | 0.63 |
| Gatifloxacin | Fluoroquinalones | 0.56 | 0.75 |
| Imipenem | Carbapenem | 0.5 | 0.63 |
| Kanamycin | aminoglycoside | 0.5 | 0.5 |
| Streptomycin | aminoglycoside | 0.63 | 0.75 |

### Example 12

### Studies on the Bacterial Activities of the Peptides of the Present Disclosure

### Bacterial Viability Assay:

Cell viability was conducted for two Gram-negative bacteria (Pa 9027 and Pa 27853). The cultures were grown overnight in TS agar and a few isolated colonies were inoculated to achieve a turbidity equivalent to 0.5 McFarland standard. The cell concentration was adjusted to 106 CFU/ml with 10 mM phosphate buffer and separated into different tubes to get final concentration of 105 CFU/ml. The peptides were added to the individual tubes to obtain concentrations of ⅛ MIC, ¼ MIC, ½ MIC, 1 MIC, 2 MIC and 4 MIC. The tubes were incubated at 37° C. for 22 h. Serial dilution was performed and 100 uL of the suspension was aliquot into MHA plate. The plates were incubated for 24 h at 37° C. for colony counting. Positive control was performed at 0 h and 22 h.

FIG. 12 shows the bacterial properties of both B2088 and B2088_{—}99 against various P. aeruginosa strains. FIG. 12 shows that the effective dose of B2088_{—}99 is significantly lower than B2088.

### Time-Kill Kinetics Assay:

The kinetics of bactericidal action was performed by the assay reported before. Briefly, few colonies of overnight grown P. aeruginosa strains were collected from tryptic soy agar plate and suspended US pharmacopeia phosphate buffer (pH 7.2). The suspension was adjusted to an initial inoculum of 10⁶ CFU/mL and incubated with various concentrations of B2088 and B2088_{—}99 at 35° C. 0.1 mL aliquots were withdrawn at various time intervals, diluted 102-104 fold using the same buffer, plated on tryptic soy agar plates and incubated at 35° C. The colonies were counted after 24 h incubation and expressed as CFU/mL. Buffer without peptides served as a positive control and % bacterial viability is estimated using the following equation:

Bacterial viability=1−(CFU/mL)_{peptide}/(CFU/mL)_{control}*100

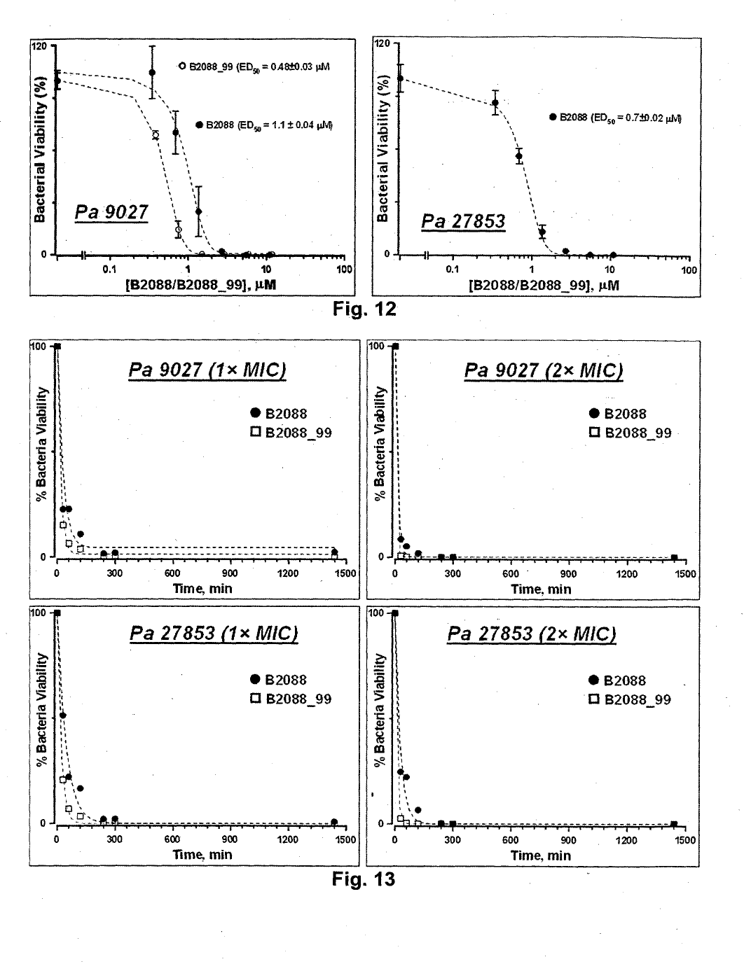

FIG. 13 shows the time-kill kinetics of B2088 and B2088_{—}99 against P. aeruginosa. B2088_{—}99 displayed faster kill kinetics against both Pseudomonas strains at 1× and 2×MIC.

### Outer Membrane (OM) Permeability Assay:

To probe the OM permeability of peptides, a membrane impermeable probe N-phenyl-1-naphthyl amine (NPN) was used in the present study. The overnight cultured P. aeruginosa ATCC 9027 cells were harvested by centrifugation at 3000 rpm, 4° C. The resulting pellet was washed twice and resuspended in 5 mM HEPES buffer (pH 7.2) to OD600 of 0.4. The cells were placed in a 10 mm stirred cuvette and NPN was added to a final concentration of 10 μM. Appropriate concentrations of B2088 was added and the increase in fluorescence intensity was monitored on a Quanta Master fluorescence spectrophotometer (Photon Technology International, New Jersey, USA). The excitation and emission wavelengths were set at 350 and 410 nm with slid widths at 2 and 5 nm, respectively. The % NPN uptake was calculated relative to increase in NPN fluorescence intensity after the addition of 50 μM polymyxin B.

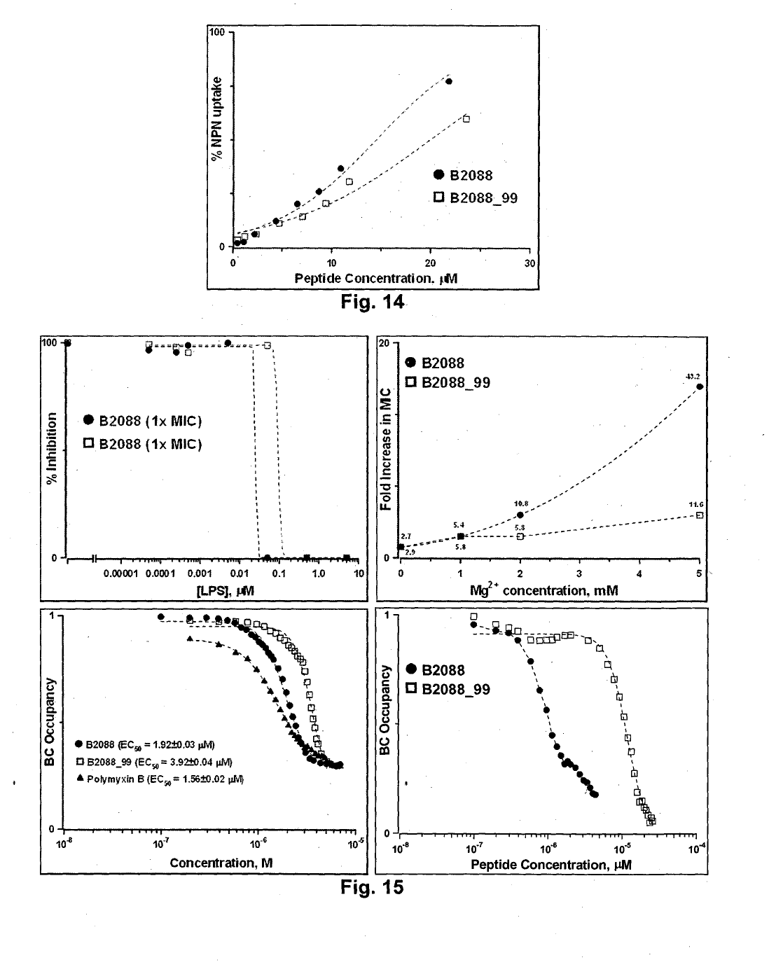

FIG. 14 shows B2088 to be more effective in causing outer membrane permeability than B2088_{—}99.

### Bodipy TR Cadavarine (BC) Displacement Assay for LPS and Lipid a Binding to Branched Peptides:

BC forms tight complex with LPS/lipid which results in quenching of its fluorescence intensity. When Peptides/molecules that can interact with LPS are added, BC is displaced from the complex with concomitant dequenching of its fluorescence. The assay was carried out in 5 mM HEPES buffer (pH 7.0). 10 μM of the dye was added to LPS or lipid A in a stirred quatz cuvette. Fluorescence measurements were performed using an excitation wavelength of 580 nm and the emission intensity at 620 nm was monitored. The displacement assay was performed by the addition of various concentrations of peptides.

Polymyxin B was used as a positive control. BC occupancy was calculated using the equation,

OF=F₀−F/F₀−Fₘₐₓ

Where F₀ is the fluorescence intensity of free BC, Fₘₐₓ is fluorescence intensity of LPS-BC complex and F is fluorescence intensity after the addition of peptides or polymyxin B. FIG. 15 shows that B2088 binds two times more strongly to lipopolysaccharides and more than 10 times more strongly to lipid A than B2088_{—}99.

In summary, the results as provided above illustrated that the removal of hydrophobic valine residues from B2088 (i.e. B2088_{—}99) lead to enhanced bactericidal and kill-kinetics properties. However, it appears that the outer membrane permeability and strong lipid A binding are compromised by the removal of hydrophobic valine residue. To confirm these results, the FIC index for B2088 and B2088_{—}99 was studied with various classes of antibiotics. As shown in the Table 8, B2088 has better sensitizing ability for various antibiotics against multidrug resistant P. aeruginosa compared to B2088_{—}99.

### Animal Model of Infection:

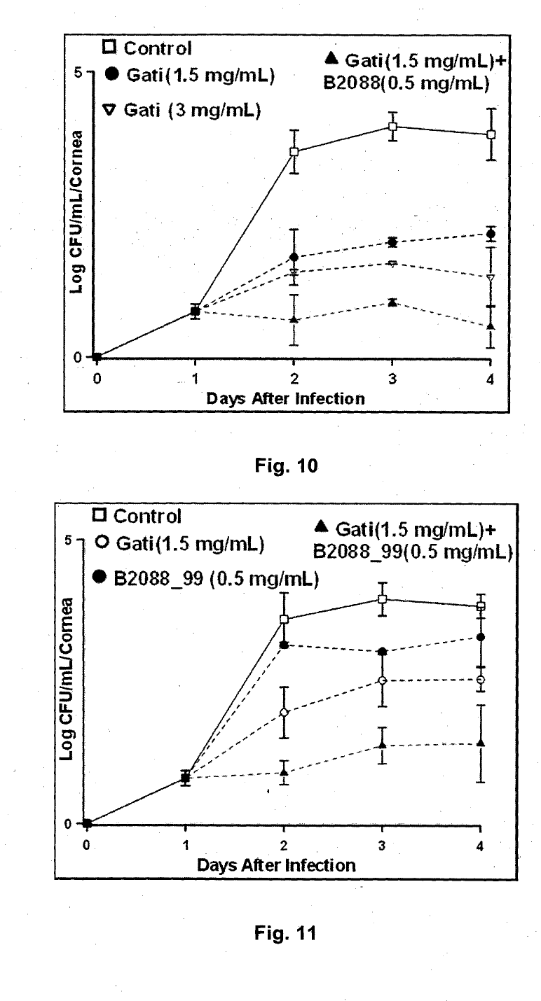

To confirm the in vitro results that B2088 has a better antimicrobial activity compared to B2088_{—}99, their ability to remove corneal infection in mouse model was investigated. The animal model of P. aeruginosa ATCC 9027 infection was used. As shown in FIG. 10, at 0.5 mg/mL of B2088 in combination with ½ the dose of gatifloxacin, a complete sterilization of the infection was observed and the activity was superior to B2088_{—}99 (FIG. 11).

1. A peptide comprising Formula I (SEQ ID NO: 1):
[(R)ₐ(X¹)_{b}(X²)_{c}(X³)ₐ(X⁴)_{b}]ₙ,
wherein X¹, X² and X⁴ are independently of each other selected from the group consisting of K, R, G and A;
X³ is K, R, L, V, I, G or A
wherein a and b are independently selected to be an integer from 1 to 10,
c is an integer selected from 0 to 5, and
n is at least one.

2. The peptide of claim 1, wherein n is any one of 1 or 2 or 3 or 4 or 5 or 6 or 7 or 8.

3. The peptide of claim 1, wherein n is at least 2 and the peptide is linked to at least two Lysine (K) residues.

4. The peptide of claim 1, wherein the peptide comprises Formula II (SEQ ID NO: 2):
[(R)ₐ(X)_{c}(K)_{b}(X)_{c}(R)ₐ(X)_{c}(K)_{b}]ₙ(K)ₙK,
wherein X is G or A, a and b is an integer selected from 1 to 10, c is an integer selected from 0 to 5 and n is at least one.

5. The peptide of claim 4, wherein the peptide of Formula II is selected from the group consisting of [(R)ₐ(X)_{c}(K)_{b}(X)_{c}(R)ₐ(X)_{c}(K)_{b}]₂KK (SEQ ID NO: 3), [(R)ₐ(X)_{c}(K)_{b}(X)_{c}(R)ₐ(X)_{c}(K)_{b}]₂KK[(K)_{b}(X)_{c}(R)ₐ(X)_{c}(K)_{b}(X)_{c}(R)ₐ (SEQ ID NO: 4), and [(R)ₐ(X)_{c}(K)_{b}(X)_{c}(R)ₐ(X)_{c}(K)_{b}]₄(K)₃K (SEQ ID NO: 5).

6. The peptide of claim 1, wherein the peptide comprises Formula III (SEQ ID NO: 6):
[R(X⁵)_{d}RK(X⁶)ₑRR]ₙ(K)ₙ₋₁K,
wherein X⁵ and X⁶ are independently of each other G, A or R, d and e are independently of each other an integer selected from 0 to 2 and n is at least one.

7. The peptide of claim 6, where X⁵ is A, the peptide comprises Formula IV (SEQ ID NO: 7):
[RARK(X⁶)ₑRR]ₙ(K)ₙ₋₁K,
wherein X⁶ is G or A, d and e are independently of each other an integer selected from 0 to 2 and n is at least one.

8. The peptide of claim 7, wherein the peptide is selected from the group consisting of (RARKAARR)₂KK (SEQ ID NO: 8) and (RARKRR)₂KK (SEQ ID NO: 9).

9. The peptide of claim 6, where X⁵ is G, the peptide comprises Formula V (SEQ ID NO: 10):
[RGRK(X⁶)ₑRR]ₙ(K)ₙ₋₁K,
wherein X⁶ is G or A, e is an integer selected from 0 to 2 and n is at least one.

10. The peptide of claim 9, wherein the peptide is selected from the group consisting of (RGRKGGRR)₂KK (SEQ ID NO: 11), (RGRKGGRR)₂KK (SEQ ID NO: 12), (RGRKGRR)₂KK (SEQ ID NO: 13), (RGRKRR)₂KK (SEQ ID NO: 14), (RGRKAARR)₂KK (SEQ ID NO: 15), (RGRKARR)₂KK (SEQ ID NO: 16), (RGRKGGRR)₂KKRRGGKRGR (SEQ ID NO: 17), (RGRKGRR)₂KKRRGKRGR (SEQ ID NO: 18) and (RGRKRR)₂KKRRKRGR (SEQ ID NO: 19).

11. The peptide of claim 6, where d and e are 0, the peptide comprises Formula VI (SEQ ID NO: 20):
[RRKRR]ₙ(K)ₙ₋₁K,
wherein n is at least one.

12. The peptide of claim 11, wherein the peptide is selected from the group consisting of (RRKRR)₂KK (SEQ ID NO: 21) and (RRKRR)₂KKRRKRR (SEQ ID NO: 22).

13. The peptide of claim 4, wherein the peptide comprises Formula VII (SEQ ID NO: 23):
[(R)ₐ(K)_{b}(X)_{c}(R)ₐ(K)_{b}]ₙ(K)ₙ₋₁K,
wherein X is G or A, a and b is an integer selected from 1 to 10, c is an integer selected from 0 to 5 and n is at least one.

14. The peptide of claim 13, wherein the peptide is selected from the group consisting of [(R)ₐ(K)_{b}X_{c}(R)ₐ(K)_{b}]₂KK (SEQ ID NO: 24), [(R)ₐ(K)_{b}X_{c}(R)ₐ(K)_{b}]₂KK[(K)_{b}(R)ₐX_{c}(K)_{b}(R)ₐ] (SEQ ID NO: 25), and [(R)ₐ(K)_{b}X_{c}(R)ₐ(K)_{b}]₄K₃K (SEQ ID NO: 26).

15. The peptide of claim 1, wherein the peptide is chemically modified.

16. The peptide of claim 15, wherein the modification is selected from the group of amidation, acetylation, stapling, replacing at least one L-amino acid with a corresponding D-amino acid, introducing or replacing at least one amino acid with a non-natural amino acid and lipidation.

17. A peptide comprising Formula VIII (SEQ ID NO: 27):
X⁷[RGRK(X⁸)(X⁹)(R)_{f}]ₙ(X¹⁰)_{g},
wherein X⁷ is a lipid group,
X⁸ and X⁹ are independently of each other, selected from the group consisting of Valine (V) and Glycine (G),
X¹⁰ is selected from the group consisting of Lysine (K) and Arginine (R),
f and g are independently of each other an integer selected from 0 to 2, and
n is at least one.

18. The peptide of claim 17, wherein the X⁷ is —RCONH, wherein R is an alkyl optionally substituted by a hydroxyl group or a carbonyl group.

19. The peptide of claim 17, wherein n is any of 1, or 2 or 3 or 4.

20. The peptide of claim 17, wherein X⁸ and X⁹ are Valine (V).

21. The peptide of claim 20, wherein the peptide is selected from the group consisting of (CH₃—CO—NH—RGRKVVRR)₂KK (SEQ ID NO: 28), (C₃H₇—CO—NH—RGRKVVRR)₂KK (SEQ ID NO: 29), (C₅H₁₁—CO—NH—RGRKVVRR)₂KK (SEQ ID NO: 30), (C7H₁₅—CO—NH—RGRKVVRR)₂KK (SEQ ID NO: 31), (C₉H₁₉—CO—NH—RGRKVVRR)₂KK (SEQ ID NO: 32), (C₁₁H₂₃—CO—NH—RGRKVVRR)₂KK (SEQ ID NO: 33), (C₁₃H₂₃—CO—NH—RGRKVVRR)₂KK (SEQ ID NO: 34) and (C₁₅H₃₁—CO—NH—RGRKVV)₂KK (SEQ ID NO: 35).

22. The peptide of claim 21, wherein the peptide is (C₇H₁₅—CO—NH—RGRKVVRR)₂KK (SEQ ID NO: 31) and (C₉H₁₉—CO—NH—RGRKVVRR)₂KK (SEQ ID NO: 32).

23. The peptide of claim 17, wherein X⁸ and X⁹ are Glycine (G).

24. The peptide of claim 23, wherein the peptide is selected from the group consisting of (CH₃—CO—NH—RGRKGGRR)₂KK (SEQ ID NO: 36), (C₃H₇—CO—NH—RGRKGGRR)₂KK (SEQ ID NO: 37), (C₅H₁₁—CO—NH—RGRKGGRR)₂KK (SEQ ID NO: 38), (C₇H₁₅—CO—NH—RGRKGGRR)₂KK (SEQ ID NO: 39), (C₉H₁₉—CO—NH—RGRKGGRR)₂KK (SEQ ID NO: 40), (C₁₁H₂₃—CO—NH—RGRKGGRR)₂KK (SEQ ID NO: 41), (C₁₃H₂₃—CO—NH—RGRKGGRR)₂KK (SEQ ID NO: 42) and (C₁₅H₃₁—CO—NH—RGRKGGRR)₂KK (SEQ ID NO: 43).

25. The peptide of claim 1, provided together with a further antimicrobial agent.

26. The peptide of claim 25, wherein the further antimicrobial agent is an antibiotic.

27-49. (canceled)

50. A kit comprising a peptide of claim 1 and instructions thereof.

51. The kit of claim 50, further comprising a second therapeutic agent.
